(19) United States
(12) Patent Application Publication
(10) Pub. No.: US 2026/0191889 A1
(43) Pub. Date: Jul. 9, 2026

(54) TREHALOSE FOR TREATING HUNTINGTON'S DISEASE

(71) Applicant: GLD Debt Acquisition 2025-1, Los Angeles, CA (US)

(72) Inventors: Raj Mehra, New York, NY (US); Krishnamurthy Ganapathy Subramanian, New York, NY (US)

(21) Appl. No.: 19/134,326

(22) PCT Filed: Nov. 29, 2023
(86) PCT No.: PCT/US2023/081629

§ 371 Date: May 30, 2025

Related U.S. Application Data
(60) Provisional application No. 63/428,796, filed on Nov. 30, 2022.
(60) Provisional application No. 63/432,633, filed on Dec. 14, 2022.

(51) Int. Cl. A61K 31/7016; A61K 31/13; A61K 31/19; A61K 31/197; A61K 31/205; A61K 31/381; A61K 31/4178; A61K 31/433; A61K 31/506; A61K 31/55; A61K 31/5513; A61K 31/7034; A61K 31/7036; A61K 31/7056; A61P 25/28

(52) U.S. Cl. CPC A61K 31/7016; A61K 31/13; A61K 31/19; A61K 31/197; A61K 31/205; A61K 31/381; A61K 31/4178; A61K 31/433; A61K 31/506; A61K 31/55; A61K 31/5513; A61K 31/7034; A61K 31/7036; A61K 31/7056; A61P 25/28

(57) ABSTRACT

The present disclosure relates to compositions comprising trehalose and methods of using same for the treatment of Huntington's disease.

### CROSS-REFERENCE TO RELATED APPLICATIONS

This application claims priority to U.S. Provisional Application No. 63/428,796, filed on Nov. 30, 2022, and U.S. Provisional Application No. 63/432,633, filed on Dec. 14, 2022, the contents of each of which are hereby incorporated by reference in their entireties.

### TECHNICAL FIELD

The present disclosure relates to compositions comprising trehalose and methods of using same for the treatment of Huntington's disease.

### BACKGROUND

Huntington's disease (HD) is a neurodegenerative disease characterized by accumulations of abnormal protein aggregates that results in a progressive decline in motor and cognitive function. It is caused by CAG repeat expansions in the huntingtin gene (HTT) that is translated into a mutant huntingtin protein (mHTT), which is prone to aggregation and is responsible for the progressive loss of neurological function.

The number of CAG repeats in the HTT gene typically ranges from 6 to 35 in healthy individuals. Individuals with 36 to 39 CAG repeats experience few to no symptoms of HD. However, individuals with 40 or more CAG repeats are nearly certain to develop HD and experience symptoms of HD. Current therapeutic strategies are confined to symptomatic treatment of the disease such as spasticity, dopaminergic agents for parkinsonian symptoms, antidepressants, and general supportive care.

### SUMMARY

Some embodiments provide a method of treating Huntington's disease in a subject in need thereof, comprising intravenously administering an aqueous pharmaceutical formulation to the subject, wherein the formulation comprises a single active ingredient consisting essentially of substantially purified trehalose (also referred to herein as molecular trehalose (MT)), wherein the subject has a Unified Huntington's Disease Rating Scale-Total Functional Capacity (UHDRS-TFC) score of 7 to 13 prior to the administration.

Some embodiments provide a method of alleviating one or more symptoms of Huntington's disease in a subject in need thereof, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of treating Huntington's disease in a subject in need thereof, comprising intravenously administering an aqueous pharmaceutical formulation to the subject, wherein the formulation comprises substantially purified trehalose and a trehalase inhibitor, wherein the substantially purified trehalose is the sole active ingredient, wherein the subject has a UHDRS-TFC score of 7 to 13 prior to the administration.

Some embodiments provide a method of alleviating one or more symptoms of Huntington's disease in a subject in need thereof, comprising intravenously administering an aqueous pharmaceutical formulation to the subject, wherein the formulation comprises substantially purified trehalose and a trehalase inhibitor as the sole active ingredients, wherein the subject has a UHDRS-TFC score of 7 to 13 prior to the administration.

Some embodiments provide a method of treating Huntington's disease in a subject in need thereof, comprising:
identifying a subject as having Huntington's disease; and
intravenously administering an aqueous pharmaceutical formulation to the subject, wherein the formulation comprises a single active ingredient consisting essentially of substantially purified trehalose, and
wherein the subject has a UHDRS-TFC score of 7 to 13 prior to the administration.

Some embodiments provide a method of treating Huntington's disease in a subject in need thereof, comprising:
identifying a subject as having Huntington's disease; and
intravenously administering to the subject an aqueous pharmaceutical formulation comprising substantially purified trehalose and a trehalase inhibitor as the sole active ingredients, and
wherein the subject has a UHDRS-TFC score of 7 to 13 prior to the administration.

Some embodiments provide a method of reducing the rate of decrease in UHDRS-TFC score in a subject having Huntington's disease, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

Some embodiments provide a method of reducing the rate of decrease in UHDRS-TFC score in a subject having Huntington's disease, comprising administering intravenously to the subject an aqueous pharmaceutical formulation comprising substantially purified trehalose and a trehalase inhibitor as the sole active ingredients; wherein the subject has a UHDRS-TFC score of 7 to 13 prior to the administration.

Some embodiments provide a method of decreasing soluble mutant huntingtin protein (mHTT) in blood plasma or cerebrospinal fluid in a subject having Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of soluble mutant huntingtin protein (mHTT) in blood plasma or cerebrospinal fluid in a subject having Huntington's disease, comprising administering intravenously to the subject an aqueous pharmaceutical formulation comprising substantially purified trehalose and a trehalase inhibitor as the sole active ingredients; wherein the subject has a UHDRS-TFC score of 7 to 13 prior to the administration.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification are incorporated herein by reference in their entireties.

The term “about” when referring to a number or a numerical range means that the number or numerical range referred to is an approximation, for example, within experimental variability and/or statistical experimental error, and thus the number or numerical range may vary up to 10% of the stated number or numerical range.

The term “acceptable” with respect to a formulation, composition or ingredient, as used herein, means having no persistent detrimental effect on the general health of the subject being treated.

The phrase “effective amount” or “therapeutically effective amount” means an amount of compound that, when administered to a subject in need of such treatment, is sufficient to (i) treat HD, (ii) attenuate, ameliorate, or eliminate one or more symptoms of HD, or (iii) delay the onset of one or more symptoms of HD, as described herein. When used in reference to treatment with more than one therapeutic agent, each agent can independently be administered in a therapeutically effective amount (e.g., an amount that would be therapeutically effective as a monotherapy) or the one or more therapeutic agents can together be a therapeutically effective amount (e.g., a therapeutically effective amount of a combination therapy) for treating HD. In other words, the amount of the individual components in a therapeutically effective amount of a combination therapy can, independently, be administered (in the combination) at less than a therapeutically effective amount when administered as a monotherapy.

As used herein, the “subject” refers to any animal, including mammals such as primates (e.g., humans), mice, rats, other rodents, rabbits, dogs, cats, swine, cattle, sheep, and horses. In some embodiments, the subject is a human. In some embodiments, the subject has experienced and/or exhibited at least one symptom of the HD to be treated.

As used herein “substantially purified trehalose”, also referred to as molecular trehalose, refers to trehalose that contains less than 1% contaminants.

As used herein the “sole active ingredient” in a formulation means that the referenced compound is the sole ingredient in a formulation that produces the intended therapeutic effect. For example, in a formulation comprising trehalose as the sole active ingredient and a trehalase inhibitor as described herein, trehalose is the sole active ingredient—the trehalase inhibitor does not produce a therapeutic effect, but rather may enhance the effect of the trehalose. Moreover, when a formulation comprises trehalose as the sole active ingredient, this phrase limits the additional components to non-active ingredients.

“Treatment” or “therapy” or “treating” (and the like) of a subject with HD refers to any type of intervention or process performed on, or the administration of an active agent (e.g., trehalose) to the subject. In some instances, treatment or therapy refers to reversing, alleviating, ameliorating, inhibiting, slowing down, or preventing the onset, progression, development, severity, or recurrence of one or more symptoms or complications, associated with HD. As used in a clinical setting, treatment and the like can include obtaining beneficial or desired clinical results and can include an improvement in the condition of a subject having HD. Beneficial or desired clinical results include, but are not limited to, one or more of the following: decreasing symptoms resulting from HD, increasing the quality of life of those suffering from HD (e.g., assessed using FACT-G or EORTC-QLQC30), decreasing the dose of other medications required to treat HD, delaying the progression of HD, and/or prolonging survival of subjects having HD. “Treatment” can also mean prolonging survival as compared to expected survival if not receiving treatment.

As used herein, “alleviate” or “alleviation,” refer to reduction, in whole or in part, of symptoms associated with HD.

### DESCRIPTION OF DRAWINGS

FIG. 1 is a graph of the cells per striatum mm² that are positive for DARPP-32 as measured by immunohistochemistry in the striatum. Data are presented as mean±SEM, n=7-12 per group. WT=wild-type, TG=transgenic R6/2 mice, MT=treated with molecular trehalose.

FIG. 2 is a graph of the cells per striatum mm² that are positive for EM48 as measured by immunohistochemistry in the cortex. Data are presented as mean±SEM, n=7-13 per group. WT=wild-type, TG=transgenic R6/2 mice, MT=treated with molecular trehalose.

FIG. 3 is a graph of the cells per striatum mm² that are positive for EM48 as measured by immunohistochemistry in the striatum. Data are presented as mean±SEM, n=7-13 per group. WT=wild-type, TG=transgenic R6/2 mice, MT=treated with molecular trehalose.

FIG. 4 is a graph of the staining percentage of the region area that are positive for GFAP as measured by immunohistochemistry in the striatum. Data are presented as mean±SEM, n=7-11 per group. WT=wild-type, TG=transgenic R6/2 mice, MT=treated with molecular trehalose.

FIG. 5 is a graph of the staining percentage of the region area that are positive for Iba-1 as measured by immunohistochemistry in the striatum. Data are presented as mean±SEM, n=7-11 per group. WT=wild-type, TG=transgenic R6/2 mice, MT=treated with molecular trehalose.

FIG. 6 illustrates the vital sign capture for IV Infusion as described herein.

### DETAILED DESCRIPTION

The present disclosure relates to compositions comprising trehalose and methods of using same for the treatment of Huntington's disease.

Clinical diagnosis of HD is typically based on confirmed family history or positive genetic test (i.e. confirmation of 36 to 39 CAG repeat s or confirmation of >40 CAG repeats); and onset of motor disturbance as defined by the Unified Huntington's Disease Rating Scale (UHDRS) total motor score (TMS) diagnostic confidence score (DCS), which ranges from 0 (no motor abnormalities suggestive of HD) to 4 (motor abnormalities>99% likely to be due to HD), wherein a score of 4 defines “motor onset” or “manifest” HD. The usual age of onset (i.e. DCS of 4) ranges between 30 to 50 years and the average duration of survival post-diagnosis is 15 to 20 years. This can also be referred to as adult onset Huntington's disease. However, juvenile onset Huntington's disease (JHD) is a form of Huntington's disease that affects children and teenagers. As used herein, “Huntington's disease” refers to both adult onset and juvenile onset Huntington's disease. In some embodiments, the HD is JHD. In some embodiments, the HD is adult onset HD.

In some embodiments, the subject is at risk of developing HD. For instance, a subject may be just under the threshold of CAG repeats (under 36 or under 40) (e.g., 25, 26, 27, 28, 29, 30, 31, 32, 33, 34, or 35 CAG repeats). In another example, the subject may exhibit one or more symptoms associated with HD (regardless of the number of CAG repeats). In these instances, methods are provided herein to treat the subject at risk of developing HD. The methods herein include treatment of a subject at risk of developing HD by intravenously administering an aqueous pharmaceutical formulation to the subject, wherein the formulation comprises a single active ingredient consisting essentially of substantially purified trehalose.

Currently, after onset, it is “function” (i.e. assessment of functional capacities), rather than motor signs, which determines disease stage of a subject (e.g. in Neurology, 1979, 29, 1-3 or in Neurology, 1981, 31, 1333-1335). The Total Functional Capacity (TFC) scale (e.g. in Movement Disorders, 1996, 11, 136-142) is a component of the UHDRS and ranges from 0 (fully dependent for all care) to 13 (fully independent) the level of independence of a person with HD (referred to herein as “UHDRS-TFC” score). This scale assesses functional status of a HD patient in terms of ability to work, handle household finances, manage domestic chores, perform activities of daily living, and level of care needed. Based on the UHDRS total functional capacity (TFC), HD is divided into stages 1 to 5 of disease progression. The categorization of HD, based on TFC score (also referred to as Shoulson and Fahn stages), are also described as early stage HD (corresponding to stages 1 or 2, based on TFC score), moderate stage or mid stage HD (corresponding to stage 3, based on TFC score) and advanced stage or late stage HD (corresponding to stage 4 or 5, based on TFC score).

Some embodiments provide a method of treating Huntington's disease in a subject in need thereof, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject in need thereof, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

In some embodiments, the one or more symptoms are selected from the group consisting of: behavioral disturbances, clumsiness, moodiness, irritability, paranoia, apathy anxiety, hallucinations, abnormal eye movements, depression, impaired ability to detect odors, dystonia, involuntary movements, trouble with balance and walking, chorea with twisting and writhing motions, unsteady gait (style of walking), slow reaction time, general weakness, weight loss, dysarthria (speech difficulties), stubbornness, rigidity (continual tension of the muscles), bradykinesia (difficulty initiating and continuing movements), severe chorea, serious weight loss, inability to speak, inability to walk, swallowing problems, inability to care for oneself, and combinations thereof.

In some embodiments, the one or more symptoms are 1-3 symptoms selected from the group consisting of: behavioral disturbances, clumsiness, moodiness, irritability, paranoia, apathy anxiety, hallucinations, abnormal eye movements, depression, impaired ability to detect odors, dystonia, involuntary movements, trouble with balance and walking, chorea with twisting and writhing motions, unsteady gait (style of walking), slow reaction time, general weakness, weight loss, dysarthria (speech difficulties), stubbornness, rigidity (continual tension of the muscles), bradykinesia (difficulty initiating and continuing movements), severe chorea, serious weight loss, inability to speak, inability to walk, swallowing problems, and inability to care for oneself.

Some embodiments provide a method of reducing behavioral disturbances in a subject with Huntington's disease comprising intravenously administering an aqueous pharmaceutical formulation to the subject, wherein the formulation comprises substantially purified trehalose as the sole active ingredient, wherein the subject has a UHDRS-TFC score of 7 to 13 prior to the administration.

Some embodiments provide a method of reducing involuntary movements in a subject with Huntington's disease comprising intravenously administering an aqueous pharmaceutical formulation to the subject, wherein the formulation comprises substantially purified trehalose as the sole active ingredient, wherein the subject has a UHDRS-TFC score of 7 to 13 prior to the administration.

Some embodiments provide a method of reducing unsteady gait in a subject with Huntington's disease comprising intravenously administering an aqueous pharmaceutical formulation to the subject, wherein the formulation comprises substantially purified trehalose as the sole active ingredient, wherein the subject has a UHDRS-TFC score of 7 to 13 prior to the administration.

Some embodiments provide a method of improving gait in a subject with Huntington's disease comprising intravenously administering an aqueous pharmaceutical formulation to the subject, wherein the formulation comprises substantially purified trehalose as the sole active ingredient, wherein the subject has a UHDRS-TFC score of 7 to 13 prior to the administration.

Some embodiments provide a method of reducing chorea in a subject with Huntington's disease comprising intravenously administering an aqueous pharmaceutical formulation to the subject, wherein the formulation comprises substantially purified trehalose as the sole active ingredient, wherein the subject has a UHDRS-TFC score of 7 to 13 prior to the administration.

Some embodiments provide a method of treating Huntington's disease in a subject determined to have Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject determined to have Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of treating Huntington's disease in a subject previously identified or diagnosed as having Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject previously identified or diagnosed as having Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of treating Huntington's disease in a subject with a clinical record indicating a diagnosis of Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject with a clinical record indicating a diagnosis of Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of treating Huntington's disease in a subject at risk of developing Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject at risk of developing Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of treating Huntington's disease in a subject suspected of having Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject suspected of having Huntington's disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

Some embodiments provide a method of alleviating one or more symptoms of Huntington's disease in a subject suspected of having Huntington's disease, comprising intravenously administering on a weekly basis an aqueous pharmaceutical formulation to the subject, wherein the formulation comprises a single active ingredient consisting essentially of substantially purified trehalose.

Some embodiments provide a method of alleviating one or more symptoms of Huntington's disease in a subject suspected of having Huntington's disease, comprising intravenously administering on a weekly basis an aqueous pharmaceutical formulation to the subject at a dose of about 90.5 mg/mL for about 60±5 minutes for volumes≤600 mL or 90±5 minutes for volumes>600 mL, wherein the formulation comprises a single active ingredient consisting essentially of substantially purified trehalose.

In any of the above embodiments, the subject can have 36 to 39 (i.e., 36, 37, 38, or 39) CAG repeats. In any of the above embodiments, the subject can have 40 or more CAG repeats.

Some embodiments provide a method of treating Huntington's disease in a subject in need thereof, comprising intravenously administering an aqueous pharmaceutical formulation to the subject, wherein the formulation comprises substantially purified trehalose and a trehalase inhibitor as the sole active ingredients, wherein the subject has a UHDRS-TFC score of 7 to 13 prior to the administration.

Some embodiments provide a method of alleviating one or more symptoms of Huntington's disease in a subject in need thereof, comprising intravenously administering an aqueous pharmaceutical formulation to the subject, wherein the formulation comprises substantially purified trehalose and a trehalase inhibitor as the sole active ingredients, wherein the subject has a UHDRS-TFC score of 7 to 13 prior to the administration.

Some embodiments provide a method of treating Huntington's disease in a subject in need thereof, comprising:
identifying a subject as having Huntington's disease; and
intravenously administering an aqueous pharmaceutical formulation to the subject, wherein the formulation comprises a single active ingredient consisting essentially of substantially purified trehalose, and
wherein the subject has a UHDRS-TFC score of 7 to 13 prior to the administration.

Some embodiments provide a method of treating Huntington's disease in a subject in need thereof, comprising:
identifying a subject as having Huntington's disease; and
intravenously administering to the subject an aqueous pharmaceutical formulation comprising substantially purified trehalose and a trehalase inhibitor as the sole active ingredients, and
wherein the subject has a UHDRS-TFC score of 7 to 13 prior to the administration.

In some embodiments, the trehalase inhibitor is selected from the group consisting of: validimycin A, trehazolin, amygdalin; or a combination of any of the foregoing; or a pharmaceutically acceptable salt of any of the foregoing.

In some embodiments, a subject can experience improved sleep quality following administration of any of the pharmaceutical formulations described herein. Accordingly, some embodiments provide a method of improving sleep quality in a subject previously identified or diagnosed has having Huntington's disease comprising intravenously administering to the subject an aqueous pharmaceutical formulation, wherein the formulation comprises a single active ingredient consisting essentially of substantially purified trehalose. In some embodiments, the methods of treating Huntington's disease described herein further comprise improving the sleep quality of the subject. As used herein, improving or improvements in “sleep quality” include, but are not limited to an increase in total sleep time, reduced sleep onset latency, improved sleep maintenance, reduced total wake time, increased sleep efficiency, and/or reduced sleep disruptive events (e.g., sleep apnea), or a combination of any of the foregoing. Sleep quality can be assessed with a variety of sleep quality tests, for example, a polysomnography test, an actigraphy test, a sleep diary, and/or a multiple sleep latency test. Sleep quality can be reported using a variety of sleep indices, for example, a Pittsburgh Sleep Quality Index (PSQI), Athens Insomnia Scale (AIS), Insomnia Severity Index (ISI), Mini-Sleep Questionnaire (MSQ), Jenkins Sleep Scale (JSS), Leeds Sleep Evaluation Questionnaire (LSEQ), SLEEP-50 Questionnaire, and/or Epworth Sleepiness Scale (ESS). See, for example, Fabbri et al. Int J Environ Res Public Health. 2021 February; 18(3): 1082. In some embodiments, a subject can experience an improved score on any of the sleep quality tests and/or any of the sleep quality indexes described herein.

In some embodiments, a second UHDRS-TFC score for the subject at a second time point is determined. In some embodiments, the second UHDRS-TFC score is 1 to 10 points higher than the first UHDRS-TFC score. In some embodiments, the second UHDRS-TFC score is 1 to 5 points higher than the first UHDRS-TFC score. In some embodiments, the second UHDRS-TFC score is 3 to 8 points higher than the first UHDRS-TFC score. In some embodiments, the second UHDRS-TFC score is 5 to 10 points higher than the first UHDRS-TFC score.

In some embodiments, the second UHDRS-TFC score is about the same as the first UHDRS-TFC score.

In some embodiments, the first UHDRS-TFC score is determined no more than seven days prior to the first administration of the pharmaceutical formulation.

In some embodiments, the second UHDRS-TFC score is determined at about 13 to about 52 weeks after the first UHDRS-TFC score.

In some embodiments, the second UHDRS-TFC score is determined at about 13, about 26, about 39, or about 52 weeks after the first UHDRS-TFC score. In some embodiments, the second UHDRS-TFC score is determined at about 4, 8, 12, or 24 weeks after the first UHDRS-TFC score.

In some embodiments, the second UHDRS-TFC score is determined about 24 weeks after the first UHDRS-TFC score. In some embodiments, the second UHDRS-TFC score is determined about 52 weeks after the first UHDRS-TFC score.

In some embodiments, the first UHDRS-TFC score is determined no more than three days prior to the first administration of the pharmaceutical formulation, wherein the second UHDRS-TFC score is determined about 24 weeks after the first UHDRS-TFC score, and wherein the second UHDRS-TFC score is greater than or equal to the first UHDRS-TFC score. In some embodiments, the first UHDRS-TFC score is determined no more than three days prior to the first administration of the pharmaceutical formulation, wherein the second UHDRS-TFC score is determined about 52 weeks after the first UHDRS-TFC score, and wherein the second UHDRS-TFC score is greater than or equal to the first UHDRS-TFC score.

In some embodiments, the second UHDRS-TFC score is from 1 to 10 points higher than the first UHDRS-TFC score. In some embodiments, the second UHDRS-TFC score is 1 to 5 points higher than the first UHDRS-TFC score. In some embodiments, the second UHDRS-TFC score is 3 to 8 points higher than the first UHDRS-TFC score. In some embodiments, the second UHDRS-TFC score is 5 to 10 points higher than the first UHDRS-TFC score.

In some embodiments, the second UHDRS-TFC score is about the same as the first UHDRS-TFC score.

In some embodiments, the second UHDRS-TFC score is determined about 13 weeks after the first UHDRS-TFC score, and wherein the second UHDRS-TFC score is greater than or equal to the first UHDRS-TFC score. In some embodiments, the second UHDRS-TFC score is determined about 24 weeks after the first UHDRS-TFC score, and wherein the second UHDRS-TFC score is about the same as the first UHDRS-TFC score.

Some embodiments provide a method of reducing the rate of decrease in UHDRS-TFC score in a subject having Huntington's disease, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

Some embodiments provide a method of reducing the rate of decrease in UHDRS-TFC score in a subject having Huntington's disease, comprising administering intravenously to the subject an aqueous pharmaceutical formulation comprising substantially purified trehalose and a trehalase inhibitor as the sole active ingredients; wherein the subject has a UHDRS-TFC score of 7 to 13 prior to the administration.

In some embodiments, a trehalase inhibitor is administered. In some embodiments, the trehalase inhibitor is selected from the group consisting of: validimycin A, trehazolin, amygdalin; or a combination of any of the foregoing; or a pharmaceutically acceptable salt of any of the foregoing.

In some embodiments, the rate of decrease in UHDRS-TFC score is reduced by about 10% to about 95%. In some embodiments, the rate of decrease in UHDRS-TFC score is reduced by about 10% to about 50%. In some embodiments, the rate of decrease in UHDRS-TFC score is reduced by about 10% to about 25%. In some embodiments, the rate of decrease in UHDRS-TFC score is reduced by about 30% to about 70%. In some embodiments, the rate of decrease in UHDRS-TFC score is reduced by about 50% to about 95%. In some embodiments, the rate of decrease in UHDRS-TFC score is reduced by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or any value in between.

In some embodiments, the second EQ-VAS score is determined after the first administration of the pharmaceutical formulation.

In some embodiments, the second EQ-VAS score is greater than the first EQ-VAS score.

In some embodiments, the first EQ-VAS score is determined prior to the first administration of the pharmaceutical formulation.

In some embodiments, the first EQ-VAS score is determined no more than seven days prior to the first administration of the pharmaceutical formulation.

In some embodiments, the first EQ-VAS score is determined no more than three days prior to the first administration of the pharmaceutical formulation.

In some embodiments, the second EQ-VAS score is determined at about 13, about 26, about 39, or about 52 weeks after the first EQ-VAS score.

In some embodiments, the second EQ-VAS score is determined at about 24 or 52 weeks after the first EQ-VAS score.

In some embodiments, the first EQ-VAS score is determined no more than three days prior to the first administration of the pharmaceutical formulation, wherein the second EQ-VAS score is determined at about 52 weeks after the first EQ-VAS score, and wherein the second EQ-VAS score is greater than the first EQ-VAS score.

In some embodiments, the second EQ-VAS score is determined about 24 weeks after the first EQ-VAS score, and wherein the second EQ-VAS score is greater than the first EQ-VAS score.

Some embodiments provide a method of decreasing soluble mutant huntingtin protein (mHTT) levels in a subject having Huntington's disease, comprising intravenously administering an aqueous pharmaceutical formulation to the subject, wherein the formulation comprises a single active ingredient consisting essentially of substantially purified trehalose, and wherein the subject has a UHDRS-TFC score of 7 to 13 prior to the administration. In some embodiments, the decrease in soluble mHTT levels in a subject comprises a reduction in soluble mHTT in the blood plasma or cerebrospinal fluid (CSF) of the subject. In some embodiments, the decrease in soluble mHTT levels in a subject is a reduction in soluble mHTT in the blood plasma of the subject. In some embodiments, the decrease in soluble mHTT levels in a subject is a reduction in soluble mHTT in the cerebrospinal fluid (CSF) of the subject.

Some embodiments provide a method of decreasing soluble mutant huntingtin protein (mHTT) levels in a subject having Huntington's disease, comprising administering intravenously to the subject an aqueous pharmaceutical formulation comprising substantially purified trehalose and a trehalase inhibitor as the sole active ingredients; wherein the subject has a UHDRS-TFC score of 7 to 13 prior to the administration. In some embodiments, the decrease in soluble mHTT levels in a subject comprises a reduction in soluble mHTT in the blood plasma or CSF of the subject. In some embodiments, the decrease in soluble mHTT levels in a subject is a reduction in soluble mHTT in the blood plasma of the subject. In some embodiments, the decrease in soluble mHTT levels in a subject is a reduction in soluble mHTT in the cerebrospinal fluid (CSF) of the subject.

In some embodiments, the soluble mHTT level is decreased by from about 25% to about 90% (e.g., from about 25% to about 90%, from about 25% to about 80%, from about 25% to about 70%, from about 25% to about 60%, from about 25% to about 50%, from about 25% to about 40%, from about 25% to about 30%, from about 30% to about 90%, from about 40% to about 90%, from about 50% to about 90%, from about 60% to about 90%, from about 70% to about 90%, or from about 80% to about 90%) after intravenous administration to the subject of an aqueous pharmaceutical formulation comprising a single active ingredient consisting essentially of substantially purified trehalose, relative to the mHTT levels prior to administration of the formulation. In some embodiments, soluble mHTT levels in blood plasma of a subject having Huntington's disease is decreased by from about 25% to about 90% following administration of any of the pharmaceutical formulations described herein. In some embodiments, soluble mHTT in CSF of a subject having Huntington's disease is decreased by from about 25% to about 90% following administration of any of the pharmaceutical formulations described herein.

In some embodiments, wild-type Huntington protein (HTT) levels are about the same before and after administration of any of the pharmaceutical formulations described herein.

Trehalose can preferentially or selectively interact with mHTT compared to wild-type HTT. Such preferential or selective interaction can decrease side effects of administration of any of the pharmaceutical formulations described herein to a subject. In some embodiments, the wild-type HTT protein is at a level and/or activity that is substantially the same before administration and after administration of any of the pharmaceutical formulations described herein. In some embodiments, the mHTT protein level is reduced more than the wild-type HTT protein level after administration of any of the pharmaceutical formulations described herein. In some embodiments, the wild-type HTT protein level is decreased less than the mHTT protein level is decreased after administration of any of the pharmaceutical formulations described herein. In some embodiments, the mHTT protein is degraded more than the wild-type HTT protein after administration of any of the pharmaceutical formulations described herein compared to before administration of the same pharmaceutical formulation. In some embodiments, the wild-type HTT protein is degraded less than the mHTT protein after administration of any of the pharmaceutical formulations described herein compared to before administration of the same pharmaceutical formulation.

Some embodiments provide a method of reducing the rate of cognitive decline in a subject having Huntington's disease, comprising intravenously administering an aqueous pharmaceutical formulation to the subject;
wherein the formulation comprises a single active ingredient consisting essentially of substantially purified trehalose.

Some embodiments provide a method of reducing the rate of cognitive decline in a subject having Huntington's disease, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

In some embodiments, the cognitive decline of a subject after intravenous administration of an aqueous pharmaceutical formulation comprising a single active ingredient consisting essentially of substantially purified trehalose for a first period of time is less than the cognitive decline of the subject for a second period of time prior to administration of the formulation.

In some embodiments, the rate of cognitive decline of a subject after intravenous administration of an aqueous pharmaceutical formulation comprising a single active ingredient consisting essentially of substantially purified trehalose for a first period of time is less than the rate of cognitive decline of the subject for a second period of time prior to administration of the formulation.

In some embodiments, the average cognitive decline of a subject after intravenous administration of an aqueous pharmaceutical formulation comprising a single active ingredient consisting essentially of substantially purified trehalose for a period of time is less than the average cognitive decline of a subject not administered the pharmaceutical formulation.

In some embodiments, the average rate of cognitive decline of a subject after intravenous administration of an aqueous pharmaceutical formulation comprising a single active ingredient consisting essentially of substantially purified trehalose for a period of time is less than the average rate of cognitive decline of a subject not administered the pharmaceutical formulation.

In some embodiments, the cognitive decline of a subject is determined by one or more scores from the Huntington's Disease Cognitive Assessment Battery (HD-CAB) as described herein.

### Huntington's Disease Cognitive Assessment Battery (HD-CAB) Test

1. Color Shape CuedSet Shifting (COSH). This computerized test assesses cued set shifting. Subjects are presented with a cue, indicating what stimulus dimension they should respond to (either “Color” or “C” for color, and either “Shape” or “S” for shape), followed by a stimulus, for example, a circle or a triangle that is either red or blue. Trials are presented in a fixed order (up to, for example, 96 trials). The outcome measure is switch cost (msec), computed using the response times for correct letter-cued trials that are preceded by correct trials. To determine the switch cost for each subject (i.e., set shifting cost), the difference between mean response times when cues changed across consecutive trials (i.e., color to shape, or shape to color) and mean response times when cues stayed the same on consecutive trials is calculated. Larger switch costs indicate poorer performance. This version is modified from a 192 trial version, which intermixed 96 trials using a 200 msec cue to stimulus delay, and 96 trials using a 1200 msec delay. To ease subject burden and make the administration time feasible in the context of a short, multi-test battery, the shortened version included only the 200 msec delay trials, which are previously found to be more sensitive.
2. Emotion Recognition (EMO). This computerized test assesses the ability to recognize facial expressions of emotion. For the test, subjects are shown a series of photographs of faces from a commonly used stimulus set. They respond by selecting the word that best describes the expression from among seven response options (anger, fear, sadness, disgust, happiness, surprise, or neutral). The test required the subject to press and hold a purple circle called the “Home key”. Pressing the home key triggers the onset of a face stimulus displaying either an emotion or a neutral expression. The face stimulus is displayed for up to four seconds. Response options, which appeared simultaneously with the face stimuli, are available for up to eight seconds. Trials end and the face and response options are replaced by a cross-hair when a subject responds or eight seconds have elapsed, whichever comes first. There can be up to 70 trials in total, with 10 of each of the six emotions, and 10 additional trials depicting neutral expressions. The outcome measure is the number of correctly identified negative emotions (anger, fear, sadness, disgust), out of 40 possible.
3. The Hopkins Verbal Learning Test Revised (HVLT-R). This is a commercially-available paper and pencil test assessing verbal learning and memory. For the test, subjects try to learn and recall a set of 12 words across three ‘learning trials’. Specifically, examiners read aloud a series of 12 words, at a pace of one every two seconds, after which subjects recalled aloud the words they could remember, in any order. Examiners then repeat the list again, in the same order, and subjects are asked again to recall all of the words they could remember, including the ones they recalled before. This process is repeated three times in total, making up the three learning trials.
4. Judgment of Line Orientation (JLOT). This computerized test assesses visuospatial perception and processing. For the JLOT, subjects view two stimulus lines at angles to each other that appeared at the top of the screen. Simultaneously, at the bottom of the screen, they view a set of response options consisting of 17 lines that are fanned out from 0 to 180 degrees in 11.25 degree increments. The 17 lines are numbered for easy identification. To respond, subjects indicate the numbers of the two lines at the bottom of the screen that exactly match the angles of the two stimulus lines at the top of the screen. The difficulty of the modified test is also enhanced by a) using shorter stimulus lines, b) using stimulus lines that are offset relative to each other rather than exactly matching the relative positioning of the response lines, c) avoiding vertical or horizontal stimulus lines and nearest neighbors of vertical or horizontal lines, and d) avoiding stimulus line pairs that are mirror reflections about the vertical. There are 20 trials. The outcome measure is the number of correct responses out of 40 (2 lines per trial).
5. The Map Search (MAP) is a subtest of the Test of Everyday Attention. MAP is a paper and pencil test assessing visuospatial attention and processing speed. Subjects are presented with a map of Philadelphia on A3 sized paper, on which are printed numerous small symbols indicating points of interest such as places to eat, petrol/gas stations, and auto repair garages. Subjects are given 60 seconds to circle (with a pen) a specified target symbol. There are three alternate forms: the Form A target is a fork and knife, the form B target is a screwdriver and spanner/wrench, and the form C target is a petrol/gas pump. Subjects completed each form only once, and form order for the three visits is counterbalanced across subjects. The outcome measure is the number of correctly circled symbols (maximum 80).
6. The Montreal Cognitive Assessment (MOCA). This is a test used to screen for mild cognitive impairment. The MOCA includes 11 subtests assessing visuospatial functions, executive function, naming, memory, attention, language, abstraction, delayed recall, and orientation. Each subtest is allotted between 1 and 5 points. CAB Beta subjects completed all subtests of the MOCA. The MOCA is scored as a total of the points earned for correct responses across all subtest scores. One point is added to the total score for individuals with education levels below 12 years, for a maximum score of 31.
7. The modified CANTAB One Touch Stockings of Cambridge (OTS). This is a computerized test assessing planning and working memory (Cambridge Cognition, Bottisham, Cambridge, UK.) For OTS, subjects are shown an image of colored balls stacked inside three ‘stockings’. A second image appears at the same time as the first, showing the same balls stacked in a different order inside the stockings. By imagining moving the balls one at a time (not actually moving them), the subject has to determine the minimum number of moves required to move the balls in the second image to match the first. Subjects respond, indicating the number of moves required, by selecting from response options 1 through 5 that are displayed at the bottom of the computer screen. If an incorrect response is selected, the subject continues attempting to solve the problem, until they determine the correct answer. The 10 trials in this test vary in difficulty, ranging from one to five moves required. The outcome measure is the mean time to reach a correct response (sec), averaged across all 10 trials. Thus, higher values indicated worse performance (longer time to a correct response).
8. Paced Tapping (PTAP). This is a computerized test used to assess timing and psychomotor coordination. Specifically, PTAP assesses the degree to which subjects can tap, using their thumbs in alternation, in a consistent, regular rhythm. Paced Tapping is administered at two rates, a faster 3 Hz rate and a slower 1.8 Hz rate, in two separate blocks of trials, with the order of the two blocks counterbalanced. A trial begins by presenting a series of tones, paced at 3 Hz or 1.8 Hz. Subjects are instructed to listen to the tone, and when ready, to begin tapping in time with the tone. Once the subject has tapped 11 times, the tone is discontinued, and subjects are to attempt to continue tapping at that same pace for 32 additional taps. Four trials are administered at each tapping speed. The main outcome is the consistency of the tapping rate, computed as the reciprocal of the standard deviation of the intertap intervals, using all taps from one or both frequency trials that occur following cessation of the pacing tones. This outcome is selected based on prior studies that demonstrated the reciprocal standard deviation of the intertap intervals is more influenced by HD status than other outcomes, such as the mean intertap interval.
9. Prospective Memory (PROM). This is a brief paper and pencil test assessing the ability to remember to perform an action, without prompting, following a delay. At the beginning of the testing session, subjects are instructed that once they came to a test that used a map, they should select a pen of a particular color from a set of three different colored pens. At each visit, a different colored pen is requested, for example, red at Visit 1, blue at Visit 2, and green at Visit 3. The outcome measure is a binomial variable ‘yes’ or ‘no’ which indicated whether they picked up the correct colored pen at the specified time, without prompting.
10. Speeded Tapping (STAP). This is a computerized test of psychomotor speed and coordination. Using a computer mouse placed in a small stabilizing platform, subjects are asked to place their non-dominant hand on the platform, in a flat position, with their index finger placed over the mouse. After the beginning of the trial is signaled by the sound of a tone, subjects are to tap as quickly as possible for 10 seconds. The end of the trial is also signaled by a tone. Up to four tapping trials are administered, all using the index finger of the non-dominant hand. The outcome measure is the mean of the intertap intervals across taps from all four trials, with higher values indicating worse performance (i.e., slower tapping).
11. Spot the Change (SPOT). This is a computerized test of visuospatial working memory. On each trial of this test, subjects view a display of five randomly placed colored squares for 250 millisecond (msec) (target display). After a 1000 msec delay (blank display), the target display is replaced with a display of five squares in the same locations, but one of the squares is encircled (response display). The non-encircled squares in the response display are the same colors as those in the target display. However, on half of the trials, the encircled square changed color. The subject is asked to respond ‘same’ if the encircled square is the same color, or ‘different’ if the color of the encircled square had changed. Subjects are instructed to respond both as quickly and as accurately as possible. There are up to 32 trials. The main outcome measure is Cowan's k²², the number of correct trials, adjusted for guessing (k=5 ([number correct hits/32]+[number correct rejections/32]−1).
12. Stroop Word Reading. This is a paper and pencil test that assesses processing and psychomotor speed. The Word Reading test is one of three components of the traditional Stroop Color Word Interference test. Subjects are shown a printed page on which the words ‘red’, ‘green’, and ‘blue’ are printed in black ink in a series of rows. They are asked to read the words in order across the rows as quickly as possible. The outcome measure from the test is the number of words correctly read in 45 seconds.
13. Symbol Digit Modalities Test (SDMT). This is a paper and pencil test assessing visuospatial attention, processing speed, and working memory. Subjects viewed a piece of paper that displayed, at the top, a ‘key’ matching a series of 9 symbols with the digits 1 through 9. The remainder of the paper shows a series of rows containing boxes in which the symbols are displayed, but the boxes below the symbols are empty. Subjects are asked to refer to the key to fill in the missing digits across the rows, as quickly and accurately as possible. The outcome measure is the number of items correctly completed in 90 seconds (maximum 110).
14. The Trail Making Test. This is a paper and pencil test assessing visuospatial attention, psychomotor speed, and flexibility. There are two conditions. For Trail Making Test A, subjects are given a sheet of paper on which the numbers 1 through 25 are displayed across the page. They are asked to connect the numbers, in ascending order, as quickly as possible. For Trail Making Test B, subjects are given a page on which both numbers (1-13) and letters (A-L) are displayed. They are instructed to connect the numbers and letters in alternation, in ascending order (i.e., 1 to A, A to 2, 2 to B, etc.). For each condition, the outcome measure is time to completion, in seconds, with longer times indicating worse performance. Each condition is limited to 300 seconds whether or not the subject had reached the final number. The value 300 is used as the data point for cases in which the subject had failed to complete.
15. The University of Pennsylvania Smell Identification Test (UPSIT). This is a paper and pencil test that assesses olfactory recognition. Test stimuli consisted of cardboard booklets with each page impregnated with a different odor. Each page also included four words that served as response options. To perform the test, the subject has to scratch the odor patch and then select the word that best described the odor. The outcome variable is the number of correctly identified odors out of, for example, 20 to 40 possible odors.

Some embodiments provide a method of stabilizing or decreasing the concentration of neurofilament light chain (NfL) polypeptide in the serum of a subject having previously been diagnosed with HD, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

Some embodiments provide a method of stabilizing the concentration of neurofilament light chain (NfL) polypeptide in the serum of a subject having previously been diagnosed with HD, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

Some embodiments provide a method of decreasing the concentration of neurofilament light chain (NfL) polypeptide in the serum of a subject having previously been diagnosed with HD, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

NfL protein is correlated with clinical severity of HD. Specifically, higher concentrations of Nfl in subject blood serum is an indicator of clinical severity of HD, and lower concentrations compared to concentrations of the same subject indicates reversal or slowing of HD progression. See, for example, Byrne et al., Sci. Transl. Med. 10, eaat7108 (2018). Concentrations of polypeptides in blood serum can be measured using a variety of assays and can be reported in picograms of polypeptide per milliliter of serum (pg/mL). For example, the concentration of NfL polypeptide can be measured using a Simoa® NF-light™ Advantage Kit by Quanterix. In some embodiments, the concentration of NfL polypeptide in serum decreases over time. In some embodiments, the concentration of NfL polypeptide in serum decreases as compared to an initial time point, such as a screening time point or a day 0 time point. In some cases, the concentration of NfL polypeptide decreases by at least 1 pg/mL (e.g., at least 2 pg/mL, at least 3 pg/mL, at least 4 pg/mL, or more) over time. In some cases, the concentration of NfL polypeptide decreases by at least 1 pg/mL (e.g., at least 2 pg/mL, at least 3 pg/mL, at least 4 pg/mL, or more) as compared to an initial time point, such as a screening time point or a day 0 time point. In some cases, the concentration of NfL polypeptide decreases by at least 1% (e.g., at least 2%, at least 3%, at least 4%, at least 5%, at least 6%, at least 7%, at least 8%, at least 9%, at least 10% or more) over time. In some cases, the concentration of NfL polypeptide decreases by at least 1% (e.g., at least 2%, at least 3%, at least 4%, at least 5%, at least 6%, at least 7%, at least 8%, at least 9%, at least 10% or more) as compared to an initial time point, such as a screening time point or a day 0 time point.

Any of the methods described herein can include administering one or more additional therapeutic agents. In some embodiments, the one or more additional therapeutic agent is administered simultaneously or sequentially in either order with the pharmaceutical formulation.

In some embodiments, the aqueous pharmaceutical formulation comprising substantially purified trehalose comprises about 5% (w/v) to about 15% (w/v) trehalose and has: a pH of about 4.5 to 7.0; less than about 0.75 endotoxin units per mL; and an osmolality of about 280 mOsm/kg to 330 mOsm/kg.

In some embodiments, the aqueous pharmaceutical formulation comprising substantially purified trehalose is formulated to be administered over about 15 minutes to about 240 minutes (e.g., about 15 minutes to about 150 minutes).

In some embodiments, the substantially purified trehalose contains less than about 1% contaminants. In some embodiments, the substantially purified trehalose contains less than about 0.5% contaminants.

In some embodiments, the pH of the formulation is about 4.5 to 7.0. In some embodiments, the pH of the formulation is about 4.5, about 4.8, about 5, about 5.3, about 5.5, about 5.8, about 6, about 6.3, about 6.5, about 6.8, about 7, or any value in between.

In some embodiments, the pH of the formulation, is about 4.4 to about 6.6. In other embodiments, the pH of the formulation, is about 4.5 to about 6.5. In still other embodiments, the pH of the formulation is about 5 to about 6. In some embodiments, the pH of the formulation is about 5.5.

In some embodiments, the formulation contains less than about 0.75 endotoxin units per mL, e.g., between an undetectable level of endotoxin units per mL, up to about 0.75 endotoxin units per mL, such as about 0.05, about 0.10, about 0.15, about 0.20, about 0.25, about 0.30, about 0.35, about 0.40, about 0.45, about 0.50, about 0.55, about 0.60, about 0.65, or about 0.70 endotoxin units per mL. In some embodiments, the formulation contains than about 0.50 endotoxin units per mL. In other embodiments, the formulation contains than about 0.25 endotoxin units per mL. In still other embodiments, the formulation contains than about 0.20 endotoxin units per mL. In some embodiments, the formulation contains about 0.01 to about 0.15 endotoxin units per mL, or any value in between. In some embodiments, the formulation contains less than about 0.15 endotoxin units per mL. In other embodiments, the formulation contains less than about 0.10 endotoxin units per mL. In still other embodiments, the formulation contains less than about 0.05 endotoxin units per mL. In some embodiments, the formulation contains less than about 0.02 endotoxin units per mL. In other embodiments, the formulation contains less than about 0.01 endotoxin units per mL. In still other embodiments, the formulation contains an undetectable amount of endotoxin units per mL. Methods for detecting endotoxin levels are known in the art, and include, for example, the rabbit pyrogen test (USP<151>), the monocyte activation test, the limulus amoebocyte lysate assay, and HPLC-based methods.

In some embodiments, the formulation has an osmolality of about 290 mOsm/kg to 320 mOsm/kg, or any value in between, for example, about 290, about 295, about 300, about 305, about 310, about 315, or about 320 mOsm/kg. In other embodiments, the formulation has an osmolality of about 300 mOsm/kg to about 310 mOsm/kg. In some embodiments, the formulation has an osmolality of about 290 mOsm/kg, about 300 mOsm/kg, about 310 mOsm/kg, or about 320 mOsm/kg.

The frequency of intravenous administration of any of the aqueous pharmaceutical formulations described herein, and optionally one or more trehalase inhibitors described herein, can be any amount that treats Huntington's disease, alleviates one or more symptoms of Huntington's disease, or reduces the rate of decrease in UHDRS-TFC score in a subject having Huntington's disease. For example, the frequency of administration of any of the aqueous pharmaceutical formulations described herein, and optionally one or more trehalase inhibitors described herein, can be from about once a day to about once a week (e.g., once every other day or once a week) or from about once a day to about once a month. For example, the frequency of administration of an aqueous pharmaceutical formulation comprising a single active ingredient consisting essentially of substantially purified trehalose can be from about once a day to about once a week (e.g., once every other day or once a week) or from about once a day to about once a month. For example, the frequency of administration an aqueous pharmaceutical formulation comprising substantially purified trehalose and a trehalase inhibitor as the sole active ingredients, can be from about once a day to about once a week (e.g., once every other day or once a week) or from about once a day to about once a month.

The frequency of intravenous administration of any of the aqueous pharmaceutical formulations described herein, and optionally one or more trehalase inhibitors can remain constant or can be variable during the duration of treatment. A course of treatment with any of the aqueous pharmaceutical formulations described herein, and optionally one or more trehalase inhibitors described herein can include rest periods in which the subject does not receive the same treatment. For example, any of the aqueous pharmaceutical formulations described herein, and optionally one or more trehalase inhibitors described herein can be administered weekly over a one to six month period followed by, for example, a one-week rest period or a one month rest period, and such a regimen can be repeated multiple times. As with the effective amount, various factors can influence the actual frequency of administration used for a particular application. For example, the effective amount, duration of treatment, use of multiple treatment agents, route of administration, and severity of Huntington's disease may require an increase or decrease in administration frequency.

An effective duration for any of the aqueous pharmaceutical formulations described herein, and optionally one or more trehalase inhibitors can be any duration that treats Huntington's disease, alleviates one or more symptoms of Huntington's disease, or reduces the rate of decrease in UHDRS-TFC score in a subject having Huntington's disease. In some cases, the effective duration can vary from several days to several months. Multiple factors can influence the actual effective duration used for a particular treatment. For example, an effective duration can vary with the frequency of administration, effective amount, use of multiple treatment agents, route of administration, and severity of Huntington's disease.

In some embodiments, the formulation is administered over about 15 minutes to about 240 minutes, or any value in between. In some embodiments, the formulation is administered over about 15 minutes to about 150 minutes. In some embodiments, the formulation is administered over about 15 minutes to about 90 minutes. In other embodiments, the formulation is administered over about 15 minutes to about 60 minutes. In other embodiments, the formulation is administered over about 15 minutes to about 45 minutes. In some embodiments, the formulation is administered over about 15 minutes to about 30 minutes. In other embodiments, the formulation is administered over about 120 minutes to about 150 minutes. In other embodiments, the formulation is administered over about 120 minutes to about 240 minutes. In other embodiments, the formulation is administered over about 150 minutes to about 240 minutes. In some embodiments, the formulation is administered over less than about 240 minutes, less than 200 minutes, less than about 150 minutes, less than about 120 minutes, less than about 90 minutes, less than about 60 minutes, less than about 45 minutes, or less than about 30 minutes.

In some embodiments, the formulation comprises about 5% (w/v) to about 15% (w/v) substantially purified trehalose, or any value in between, for example, about 5, about 6, about 7, about 8, about 9, about 10, about 11, about 12, about 13, about 14, or about 15% (w/v) substantially purified trehalose. In some embodiments, the formulation comprises about 6% (w/v) to about 12% (w/v) substantially purified trehalose. In other embodiments, the formulation comprises about 7% (w/v) to about 11% (w/v) substantially purified trehalose. In still other embodiments, the formulation comprises about 8% (w/v) to about 10% (w/v) substantially purified trehalose. In some embodiments, the formulation comprises about 9% (w/v) substantially purified trehalose. In other embodiments, the formulation comprises about 10% (w/v) substantially purified trehalose. In still other embodiments, the formulation comprises about 8% (w/v) substantially purified trehalose.

In some embodiments, the substantially purified trehalose contains less than about 0.5% contaminants, e.g., an undetectable level of contaminants by HPLC to about 0.5% contaminants, or any value in between. In some embodiments, the substantially purified trehalose contains about 0.01, about 0.02, about 0.03, about 0.04, about 0.05, about 0.08, about 0.10, about 0.12, about 0.15, about 0.18, about 0.20, about 0.22, about 0.25, about 0.28, about 0.30, about 0.32, about 0.35, about 0.38, about 0.40, about 0.42, about 0.45, about 0.48, or about 0.5% contaminants. In other embodiments, the substantially purified trehalose contains less than about 0.25% contaminants. In still other embodiments, the substantially purified trehalose contains less than about 0.1% contaminants. In some embodiments, the substantially purified trehalose contains less than about 0.08% contaminants. In other embodiments, the substantially purified trehalose contains less than about 0.05% contaminants. In still other embodiments, the substantially purified trehalose contains less than about 0.02% contaminants. In some embodiments, the substantially purified trehalose contains less than about 0.01% contaminants. In some embodiments, the contaminants in the substantially purified trehalose comprise glucose, maltotriose, or a combination thereof. In some embodiments, the formulation contains less than about 0.01% glucose and less than about 0.01% maltotriose.

Some embodiments further comprise administering a trehalase inhibitor as a non-fixed combination with the pharmaceutical formulation comprising a single active ingredient consisting essentially of substantially purified trehalose. In some embodiments, the trehalase inhibitor is selected from the group consisting of validimycin A, trehazolin, amygdalin; or a combination of any of the foregoing; or a pharmaceutically acceptable salt of any of the foregoing. In some embodiments, the trehalase inhibitor is administered simultaneously or sequentially in either order with the pharmaceutical formulation comprising a single active ingredient consisting essentially of substantially purified trehalose.

Some embodiments further comprise monitoring pharmacokinetic values following administration of any of the pharmaceutical compositions described herein. For example, the area under the curve (AUC), maximum concentration (Cₘₐₓ), time to maximum concentration (Tₘₐₓ) and terminal half-life (t_{1/2}) on, for example, day 1 and week 12 of administration of the pharmaceutical composition.

Some embodiments further comprise monitoring huntingtin protein (HTT) levels. Non-limiting examples of how HTT levels can be monitored include the amount of HTT (native HTT, total HTT, and/or mHTT) in biofluids (e.g. blood (e.g., plasma) and/or cerebrospinal fluid (CSF)),

Some embodiments further comprise assessing known biomarkers of Huntington's Disease, including but not limited to mutant huntingtin protein (mHTT) levels, native huntingtin protein (HTT) levels, or total huntingtin protein (HTT) levels, e.g., in blood (e.g., plasma) and/or CSF. For example, a reduction in soluble or a reduction in total HTT levels in CSF and/or blood (e.g., plasma) can be observed.

Monitoring or assessing protein biomarkers can use any known protein monitoring or assessment method including western blot, immunoblot, mass spectrometry, SDS-page gel analysis, and the like.

### EXAMPLES

### Example 1: Testing Efficacy of Molecular Trehalose (MT) in R6/2 Mouse Model of Huntington's Disease

The objective of this study was to assess the effects of molecular trehalose (MT) in the R6/2 mouse model of Huntington's disease through intraperitoneal (IP) injections. Treated mice (n=7) were administered (i.e. dosed) with a MT intraperitoneal (i.p.) injection. Mice were weighed twice per week until 8 weeks post infusion i.e., 12 weeks of age or followed until survival. At a minimum of 8 weeks post infusion or MT dosing, mice were sacrificed 30 minutes after last MT, and the following tissues were collected: blood, brain, and tail. Immunohistochemistry analysis for GFAP, Iba1, EM-47 and Darpp-32 were performed on brain hemisphere 1 samples and the amount of MT in blood and brain hemisphere 2 samples was determined.

### Materials

### Animals and Animal Husbandry

Male R6/2 (B6CBA-Tg(HDexon1)62Gpb/3J) (The Jackson Laboratory, USA, JAX stock: 006494) mice (Mus musculus) between the ages of 28-33 days were used in in this study. Mice were social house with 2-5 mice per cage with individual ventilation caging systems in polycarbonate Type II long cages (Allentown Inc.) Environmental enrichment was provided as was a plastic igloo for shelter, gnawing material (aspen sticks), and nesting material (corn cob bedding). Mice were fed a standard chow of Teklad Global 2016 pellet Ad libitum. Softened chow was be provided on cage floor to all mice and replaced daily. In addition, diet gel 76A, Wheat, Clear H₂O was provided on cage floor to all mice and replaced as necessary (at least twice per week). Tap water was provided Ad libitum. Room temperature was 21.5±1.5° C. Room lights were on from 7 AM to 8 PM. Mice were only handled together with husbandry routines prior to beginning of the study in vivo activities.

### Molecular Trehalose (MT) Preparation

Trehalose dihydrate was dissolved in water for injection once a week. Correction factor 1.105 was applied when preparing trehalose dihydrate solution to achieve the dose 5 g/kg of pure molecular trehalose. Heating was used if needed. After clear solution was achieved, MT formulation was divided into daily aliquots in plastic Eppendorf tubes and stored at 4° C. until dosing. Thirty minutes before dosing MT formulation was warmed to room temperature.

### Experimental Design

### Experimental Groups

Before the initiation of in-life phase, any animals considered unsuitable for use in the study may be replaced by alternate animals obtained from the same shipment and maintained under the same environmental conditions. Animals will be assigned to groups by a stratified randomization scheme designed to achieve balanced groups based on bodyweight.

Eleven male mice were treated with trehalose 5 g/kg (i.p. injection, 25 ml/kg, trehalose dose formulation concentration: 0.2 g/ml, with active trehalose corresponding to 5.252 g/kg of trehalose dihydrate), 5 times per week starting at 4 weeks of age. There were a total of 41 doses or doses stopped when mice were euthanized. Mice were euthanized at 8 weeks after first trehalose dose (12 weeks of age, n=8). n=3 mice/group were monitored until survival.

### Methods

### In Life Measurements and Sampling

Assessment of the presence of portosystemic shunting in mice by plasma bile acid level measurement (Main Study and Survival Study Arm)—Portosystemic shunts have been identified in significant percentage of C57BL/6J mice, resulting in major alteration in brain morphometry, brain metabolites, physiological readouts (e.g. body weight, liver enzymes), and cognitive deficits. Prior to study start, plasma bile acid analysis were performed to exclude animals with abnormally high bile acid concentration (>20 μmol/L, in addition cohort-by-cohort evaluation of the data for possible analytical shifts) which is a surrogate marker of portosystemic liver shunt. See, for example, Cudalbu et al. 2013. Blood samples for bile acid analysis were collected from saphenous vein into Li-Hep tubes. Plasma was separated by centrifugation at 2000 g (4° C.) within 30 minutes from collection. About 30 pd of Plasma was aliquoted into pre-cooled polypropylene tubes and stored at −80° C. until analysis. Plasma bile acid concentrations were analyzed using a Thermofisher Konelab Xti 20 analyzer according to manufactures instructions.

Body Weights—Body weights were monitored twice per week throughout the study for all animals. Pre-trehalose treatment onset baseline body weight was used to assign the animals to experimental groups. Terminal body weights were not registered from animals found dead or euthanized moribund.

Drug Delivery—Administration of MT was performed by investigators blinded to the treatment.

### Molecular Trehalose Administration

MT was administered to mice i.p. 5 times per week (from Monday to Friday). Dose volume was 25 ml/kg. Dose was 5 g/kg. Pure trehalose dose formulation concentration was 0.2 g/ml. The last dose of animals euthanized at 8 weeks post infusion (age week 12) was 30 min prior to terminal sampling.

Sample Collection (n=8/Group)

At 8 weeks post infusion or trehalose dosing start (12 weeks of age), n=8/group (Groups 1-4) or at 4 weeks post infusion (8 weeks of age) 1 mouse (group 5), mice had their terminal body weight recorded and were euthanized by deeply anesthesia with pentobarbital (180 mg/kg, i.p.) 30 min post last vehicle/trehalose dose.

Next, blood was collected via cardiac puncture and plasma was separated. The animals were transcardially perfused with ice cold heparinized (Heparin 2.5 IU/ml) saline. Next, brain samples were collected and processed for QuantiGene and IHC analysis.

Blood sample processing—Preparation of Solution. An acidification solution of formic acid 5% in Milli-Q Type Water (5:100) was made. In a 25 mL glass bottle, 20 mL of Milli-Q type water or HPLC grade water using a graduated cylinder was added to 1 mL of formic acid using borosilicate pipetor by adding the contents of a commercially available 1 mL ampule of formic acid. The solution was mixed well and stored at room temperature for a maximum of one month.

Study sample handling—0.5 mL of blood was collected into labelled microcontainer tubes containing EDTA K2 and immediately put into an ice-water bath. The microcontainer tubes were centrifuged at 2000×g for 10 minutes at 4° C. as soon as possible following collection, and within one hour recommended. The microcontainer tubes were put into an ice-water bath.

Preparation of study samples for analysis—10 μL of acidification solution was added to a labelled polypropylene cryovial. 50 μL of plasma was transferred into the labelled polypropylene cryovial containing 10 μL of acidification solution and cap the tubes. Volumes were modified to ensure that the plasma/acidification solution ratio remained the same. Each polypropylene tube adequately for 10 seconds using a vortex mixer. The polypropylene cryovials were stored at −80° C. until sample analysis was completed.

### Ex Vivo Samples Analysis

Cryosectioning—Cryosectioning was performed to striatal (left hemisphere) (Bregma +1.30 mm to −0.8 mm from Bregma, containing the infusion site at +0.5 mm) region and sections were collected as 20 μm thick sections on slides. A series of sections consisted of 8 sections with an interval of 300 μm in-between each section. A total of 5 series were collected from the target region using subsequent slides. For phase I, Series 1 & 3 were used for IHC analysis of EM48, series 2 was used for Darpp-32, series 4 was used for Iba1+GFAP and Series 5 was used for piloting and negative controls. For phase II, A total of 6 series were collected from the target region using subsequent slides. Series 1 was used for IHC analysis of EM48, series 2 was used for Darpp-32, series 4 was used for Iba1+GFAP. Series 5 was collected as spare samples and series 6 was used for piloting and negative controls.

### Chromogenic Detection/Immunostaining—

The immunohistochemical staining listed below were performed for the cryosectioned slides.

The used antibodies/reagents were:
mouse monoclonal IgG against huntingtin protein, clone mEM48 (Millipore, cat. no. MAB5374) (dilution 1:400)
rabbit polyclonal IgG against dopamine- and cAMP-regulated neuronal phosphoprotein (DARPP-32; Millipore/Sigma-Aldrich, cat. no. AB10518) (dilution 1:5000)
chicken polyclonal antibody against glial fibrillary acidic protein (GFAP; Abcam, cat. no. ab4674) (dilution 1:250)
rabbit polyclonal IgG against ionized calcium-binding adaptor protein-1 (Iba-1; Wako #019-19741) (dilution 1:500)

For fluorescence immunohistochemistry (GFAP and Iba-1) the following general protocol was applied:

Cryosections were taken to room temperature from storage, dried and rehydrated. Antigen retrieval was performed, where appropriate (for both GFAP and Iba-1). The sections were permeabilized followed by blocking against non-specific binding. The sections were then incubated for overnight in the primary antibody solution. The following day, the sections were washed and incubated in the appropriate secondary antibody for 2 h at RT. (Autofluorescence was blocked using 0.3% Sudan Black B in 70% ethanol.) After washing, the sections were mounted with a water-soluble mounting medium containing 4′-6-Diamidino-2-phenylindole (DAPI) to counterstain the nuclei, coverslipped, cured, sealed and stored at +4° C. until imaged.

For brightfield immunohistochemistry the following general protocol was applied (EM48 and DARPP-32):

Cryosections were taken to room temperature from storage, dried and rehydrated. Endogenous peroxidase activity was blocked, and the sections were carefully washed and permeabilized, and blocked against non-specific binding, followed by an overnight incubation in the primary antibody solution. The following day the sections were washed and incubated in the appropriate secondary antibody (biotinylated) for 2 h at RT. After washing, the sections were incubated with the tertiary reagent (avidin-biotin complex; ABC), washed and incubated with the 3,3′-diaminobenzidine (DAB) chromogen to reach a desired staining intensity (4 min; exactly same conditions for each slide). Finally, the sections were dehydrated, mounted with an organic solvent based mounting medium and coverslipped. The slides were dried and stored at RT until imaged.

Antigen expression analyses—The stained sections were scanned with the Olympus VS120 slide scanner using the proper fluorescent or brightfield channels. The regions of interest (ROIs) (striatum/cortex) were analyzed for positive immunostaining of the respective targets (EM48, DARPP-32, GFAP and Iba1) applying the VIS—Visiopharm Integrator System (Visiopharm, Denmark).

Magnification used for the scanning and exposure times was decided after initial screenings of the stainings.

Positive staining of the respective antigens was detected by applying a suitable threshold which recognizes only truly positive staining. Depending on the antigen, the results are presented as either intensity or percent positive area or the number of positive cells within the ROIs (striatum/cortex), whose total area is also measured (percent positive area=stained area within the ROI/total ROI area*100).

For the individual stainings, the assays were performed as follows:
GFAP: % staining area
Iba-1: % staining area
EM-48: number of positive cells/mm² (striatum and cortex)
DARPP-32: number of positive cells/mm² (striatum)

QuantiGene gene expression assays—The expression of 4 target mRNAs were examined in the striatum (right hemisphere, total n=33) using the branched DNA assay (QuantiGene Plex, Invitrogen). To prepare the tissues for QuantiGene analysis, the tissues (5-10 mg pieces) were homogenized using the QuantiGene Sample Processing Kit for Fresh or Frozen Tissues according to the manufacturer's protocol using TissueLyser II and 5 mm steel beads (Qiagen). The cleared lysates were used in the QuantiGene gene expression assay.

Briefly, gene expression levels were determined using custom-prepared QuantiGene Plex sets and a QuantiGene Plex Assay Kit. The assay was performed according to the instructions provided by the manufacturer (Invitrogen). A small pilot was run separately before the actual assays to determine the optimal sample input to be used in the assays. Target expression levels were normalized using the geometrical mean of at least three different housekeeping genes identified in Table 1.

Mouse gene products to be measured by the QuantiGene (bDNA) method (8plex). Housekeeping genes are marked (*).

TABLE 1
QuantiGene Gene Expression Assay Gene Targets

| Target | | Gene ID | Accession |
|---|---|---|---|
| symbol | Name | (Mus musculus) | No. |
| HTT_{—} | HTT (KI) or Htt_knock_in | | GS00936 |
| knock_in | or KI mHTT | | |
| Darpp-32 | Protein phosphatase 1 | 19049 | NM_144828 |
| IL-1b | Interleukin 1 beta | 16176 | NM_008361 |
| TNF-a | Tumor necrosis factor | 21926 | NM_013693 |
| | (alpha) | | |
| Atp5b* | ATP synthase, H+ | 11947 | NM_016774 |
| | transporting mitochondrial | | |
| | F1 complex, beta subunit | | |
| Eif4a2* | Eukaryotic translation | | NM_013506 |
| | initiation factor 4A2 | | |
| Rpl13a* | Ribosomal protein L13a | | NM_009438 |
| Canx* | Calnexin | | NM_007597 |

*indicates housekeeping genes.

Trehalose Assay—Trehalose measurements were taken across the entire brain hemisphere 2 for all mice. The hemisphere was divided into anterior, middle, and posterior thirds and the trehalose concentration was measure in each third.

Statistical Analysis and Data Graphical Representation Prior to further statistical analysis, data quality check and validations was performed. During that process, potential outliers were identified and assessed. No outliers were removed from data without a clear justification for removal (e.g., identified measurement error in the laboratory notes).

Assumption of normality of each data set was primarily based on experience (e.g., data within a population is known to be approximately Gaussian) and observations during validation phase. Some biological variables are known to follow lognormal distributions—in these cases the data were first transformed to logarithms and then parametric statistical test were used. The same normality assumption were used for series of experiments, or group of similar readouts from an assay. In addition, the D'Agostino-Pearson omnibus normality test was used to support the decision whether parametric or nonparametric test should have been used. A summary of readouts for statistical analysis and visualization is shown in Table 2.

TABLE 2
Statistical Analysis and Visualization Summary.

| Test/Assay | Normalization | Visualization |
|---|---|---|
| BW | no | line (by group + by time) |
| Survival | no | Survival curve |
| QuantiGene | Targets normalized against | scatter |
| | the housekeeper gene(s) | |
| IHC analysis | no | scatter |

Body Weight—Comparisons between two groups used a two-way Mixed-effects model (Mixed Anova) with Geisser-Greenhouse correction (not assuming sphericity), followed by Fisher's LSD test between the two groups performed for each time point. Time, group and the interaction of Time×Group were fixed effects, and the individual subject was the random effect. In cases of longitudinal comparison of three or more groups, similarly, the two-way Mixed-effects model with Geisser-Greenhouse correction, followed by Dunnett's multiple comparisons test was performed. In this case, one family per time point will be set. Time, group and the interaction of Time×Group were fixed effects, and the individual subject was the random effect.

QuantiGene, trehalose assay and IHC—Simple comparisons between two groups (disease model assessment) used unpaired Welch's t-test, or, when the assumption of normality or log-normality was not met, by the Mann-Whitney U-test. Comparison involving three or more groups used the one-way ANOVA followed by Dunnett's multiple comparisons test (treatment vs. control). In case of significantly different standard deviations detected by Brown-Forsythe test, the assumption of equal standard deviations is rejected and Welch's ANOVA followed by Dunnett's T3 multiple comparison test was used. The non-parametric counterpart is the Kruskal-Wallis test followed by Dunn's multiple comparison test.

All values were presented as the mean±standard error (SE) of the mean. In case of two or more comparisons per family, the multiplicity adjusted P values are presented. All statistical analyses were conducted with a significance level of α=0.05, using GraphPad Prism (Version 8.3, GraphPad Software, Inc., San Diego, CA) or the R statistical software environment (R: A Language and Environment for Statistical Computing; R Core Team/R Foundation for Statistical Computing; Vienna, Austria; 2019; R-project.org).

### Results

Measurement of Mouse Lifespan after Treatment with MT

To determine the effect of MT treatment on lifespan, transgenic R6/2 mice (a disease model for Huntington's Disease) were treated with MT as described above. Controls were transgenic R6/2 mice that did not receive MT treatment and wild-type mice. Wild-type mice (n=12) had a median lifespan of 181 days. R6/2 mice treated with vehicle (n=12) had a decreased median lifespan of 101, showing that a severe Huntington's disease mouse model was established. R6/2 mice treated with MT had a median lifespan of 119 days. The R6/2 mice treated with MT had a partially restored lifespan compared to wild-type mice.

Measurement of Mouse Body Weight after Treatment with MT

To determine the effect of MT treatment on body weight, transgenic R6/2 mice (a disease model for Huntington's Disease) were treated with MT as described above. Controls were transgenic R6/2 mice that did not receive MT treatment and wild-type mice. Data were reported as average±standard error. Wild-type mice (n=12) had an average final body weight of 30.7 g±0.8. R6/2 mice treated with vehicle (n=12) had a decreased average final body weight of 16.5 g±0.4, showing that a severe Huntington's disease mouse model was established. R6/2 mice treated with MT had an average final body weight of 21.5 g±0.6. The R6/2 mice treated with MT had a partially restored average final body weight compared to wild-type mice.

Measurement of DARPP-32, EM48, GFAP, and Iba-1 in the Brain Using IHC after Treatment with MT

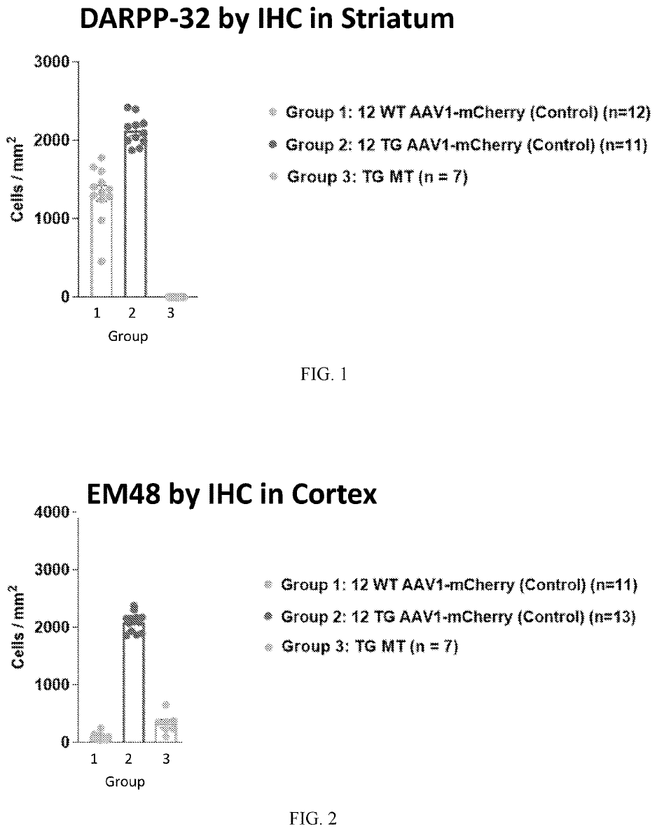

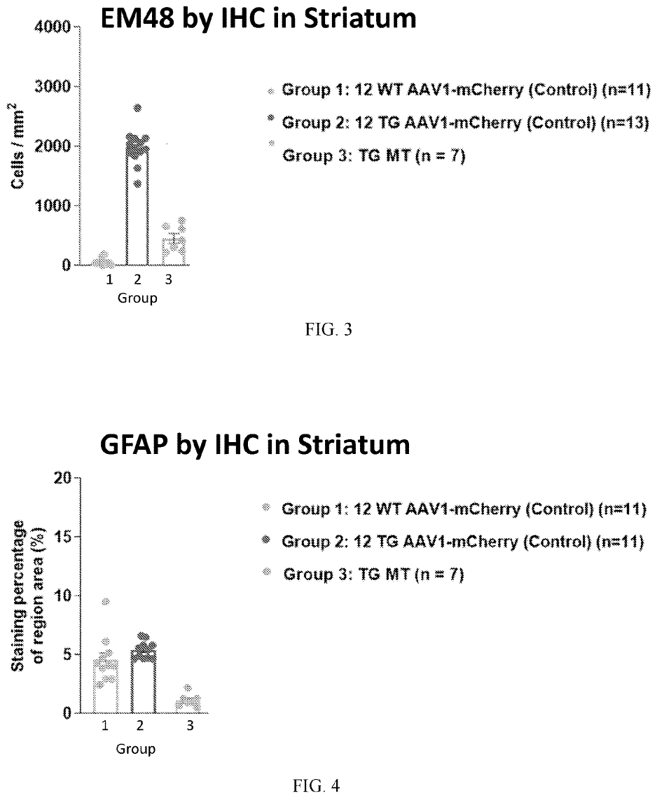

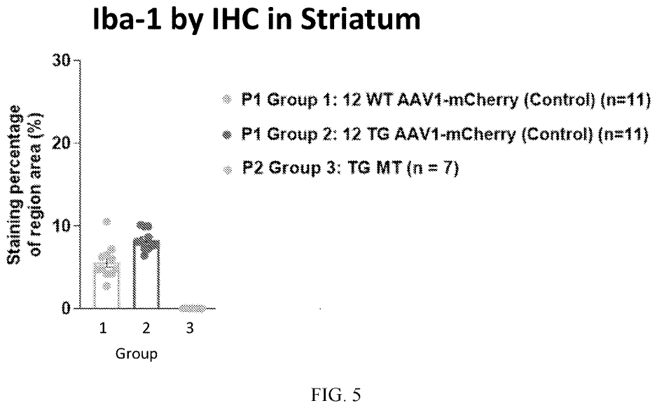

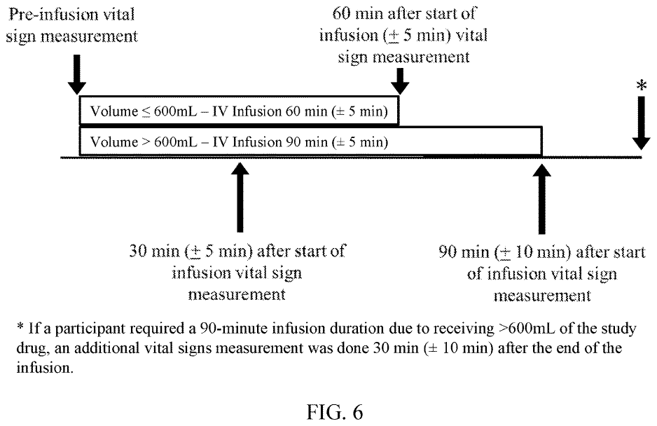

To determine the effect of MT treatment on DARPP-32, EM48, GAFP, and Iba-1, transgenic R6/2 mice (a disease model for Huntington's Disease) were treated with MT as described above. Brain samples were collected, and DARPP-32, EM48, GFAP, and Iba-1 were measured in the striatum or cortex using IHC analysis. Treatment of R6/2 mice with MT resulted in lower levels of DARPP-32 in the striatum (FIG. 1), lower levels of EM48 in the cortex and striatum (FIG. 2 and FIG. 3), lower levels of GFAP in the striatum (FIG. 4), and lower levels if Iba-1 in the striatum (FIG. 5) as compared to untreated transgenic R6/2 mice. Therefore, mHTT aggregates in the striatum and in the cortex of the brain of mice treated with MT were reduced compared to mHTT aggregates in the striatum and in the cortex of the brain of mice that were not treated with MT.

### Example 2. A 24-Week Open-Label Phase 2 Study of Trehalose Injection, 90.5 Mg/mL for Intravenous (IV) Infusion in the Treatment of Adults with Huntington's Disease (HD)

This example describes a 24-week open-label Phase 2 study of trehalose injection, 90.5 mg/mL for intravenous infusion in the treatment of adults with Huntington's disease (HD).

### Study Overview:

This is an open-label study of trehalose injection, 90.5 mg/mL for intravenous (IV) infusion for treatment of adults with HD. The Phase 2 study consists of a 2-week screening period, a 24-week open-label treatment period, and a 2-week safety follow-up period. Eligible participants will receive trehalose injection, 90.5 mg/mL for intravenous (IV) infusion administered as a once weekly IV infusion at a dose of 0.75 g/kg. The Phase 2 study will enroll 5-15 participants with HD.

### Number of Participants:

The Phase 2 study will enroll 5-15 participants with HD.

### Objectives:

### Primary Safety Objective:

The primary safety objective of the study is to determine the safety and tolerability of trehalose injection, 90.5 mg/mL for intravenous infusion in participants with HD.

### Primary Therapeutic Objective:

The primary therapeutic objective is to determine treatment responses of trehalose injection, 90.5 mg/mL for intravenous infusion in participants with HD using a patient-reported outcome measure.

### Secondary Objective:

The secondary objective is to determine the effectiveness of trehalose injection, 90.5 mg/mL for intravenous infusion on disease progression and severity in participants with HD using clinician and patient-reported outcome measures.

### Exploratory Objectives:

The exploratory objectives are:
(1) Assess the pharmacokinetics (PK) of trehalose injection, 90.5 mg/mL for intravenous infusion in participants with HD; and
(2) Assess the effect of trehalose injection, 90.5 mg/mL for intravenous infusion on neurofilament light chain concentration in blood.
(3) Assess known biomarkers of Huntington's disease including mutant huntingtin protein (mHTT) levels, native huntingtin protein (HTT) levels, or total huntingtin protein (tHTT) levels in blood (e.g., plasma) and/or cerebrospinal fluid (CSF).

### Criteria for Evaluation:

### Primary Safety Endpoint:

The primary safety endpoint will assess incidences of treatment-emergent adverse events (TEAEs) and serious adverse events (SAEs), including clinically significant laboratory or diagnostic testing abnormalities.

### Primary Therapeutic Endpoint:

The primary therapeutic endpoint is the Treatment Response Rate—Participant (TRR-P) at week 24 [Percentage of participants reporting a treatment response defined as improvement or no change from baseline on the Patient Global Impression of Change (PGIC)].

### Secondary Therapeutic Endpoints:

The secondary therapeutic endpoints are:
(1) TRR-P at weeks 4, 8, 12 and 18;
(2) Treatment Response Rate—Investigator (TRR-I) at weeks 4, 8, 12, 18 and 24 [Percentage of investigators reporting a treatment response defined as participant improvement or no change from baseline on the Clinical Global Impression of Change (CGIC)];
(3) Improvement Rate—Participant (IR-P) at weeks 4, 8, 12, 18 and 24 [Percentage of participants reporting improvement from baseline on the Patient Global Impression of Change (PGIC)];
(4) Improvement Rate—Investigator (IR-I) at weeks 4, 8, 12, 18 and 24 [Percentage of investigators reporting participant improvement from baseline on the Clinical Global Impression of Change (CGIC)];
(5) Change from baseline in Patient Global Impression of Severity (PGIS) at weeks 4, 8, 12, 18, and 24;
(6) Change from baseline in Clinical Global Impression of Severity (CGIS) at weeks 4, 8, 12, 18, and 24;
(7) Change from baseline in EuroQol visual analogue scale (EQ VAS) score at weeks 4, 8, 12, 18 and 24;
(8) Change from baseline in disease specific scale score cohort at weeks 4, 8, 12, 18 and 24;
   a. HD—Unified Huntington Disease Rating Scale (UHDRS)—Total Functional Capacity (TFC) and Independence Scale.

### Exploratory Endpoints:

Change from baseline in blood neurofilament light chain concentrations at weeks 12 and 24; and serum PK parameter estimates for trehalose injection, 90.5 mg/mL for intravenous infusion including, but not limited to, the area under the curve (AUC), maximum concentration (Cₘₐₓ), time to maximum concentration (Tₘₐₓ) and terminal half-life (t_{1/2}) on day 1 and week 12. As briefly described above, this is an open-label study of trehalose injection, 90.5 mg/mL for intravenous infusion for treatment of adults with HD. The Phase 2 study consists of a 2-week screening period, a 24-week open-label treatment period, and a 2-week safety follow-up period. Eligible participants will receive trehalose injection, 90.5 mg/mL for intravenous infusion to be administered as at least once weekly and up to multiple times a day IV infusion over 60 minutes±5 min for volumes≤600 mL or 90 minutes±5 min for volumes>600 mL. The total volume of the infusion will be based on participant weight and dose of 0.75 g/kg. The study will enroll 5 to 15 participants with HD. Furthermore, the primary study objectives are to determine safety and tolerability and treatment responses associated with trehalose injection, 90.5 mg/mL for intravenous infusion in participants with HD. Safety and tolerability will be determined by incidences of treatment-emergent adverse events (TEAEs) and serious adverse events (TESAEs), including clinically significant laboratory or diagnostic testing abnormalities. Laboratory samples, ECG's and C-SSRS are to be obtained at the screening/baseline and week 4, 12 & 24 visits. Physical and neurological examinations are to be conducted at the screening/baseline and week 12 & 24 visits. Vital signs and AEs are to be obtained at visits for trehalose injection, 90.5 mg/mL for intravenous infusion infusions and any unscheduled visits. In addition, AEs may be obtained by scheduled or unscheduled telephone communications. Treatment responses will be determined by assessments and comparative analyses of the following general and disease-specific clinical measures that are scheduled to be obtained at the screening/baseline and several specified study visits.

All participants are scheduled to complete the PGIC, and all investigators are to complete the CGIC & CGI-S for the respective participant at the week 4, 8, 12, 18 & 24 visits. In addition, all participants are scheduled to complete the PGI-S and EQ-VAS and all investigators are scheduled to complete the CGI-S at the day 1 and week 4, 8, 12, 18 & 24 visits. At the screening/baseline and week 4, 8, 12, 18, & 24 visits, the Independence Scale for participants with HD will be completed.

Blood samples for measurement of neurofilament light chain concentration are scheduled to be obtained from all participants at the screening/baseline and week 12 & 24 visits for analyses of change after initiation of treatment. In addition, blood samples are scheduled to be obtained from all participants at several timepoints during the day 1 and week 12 visits to assess PK of trehalose injection, 90.5 mg/mL for intravenous infusion.

An additional 52 weeks of open-label trehalose injection, 90.5 mg/mL for intravenous infusion treatment is planned for consenting participants who are determined to be medically appropriate after their completion of the primary 24-week open-label treatment period. Participants who do not complete the primary 24-week treatment period or who completed the 24-week treatment period but do not consent to additional treatment are to be scheduled for a safety follow-up visit within 2-4 weeks of their last trehalose injection, 90.5 mg/mL for intravenous infusion dose. All participants who discontinue treatment early and do not plan to continue attending scheduled study visits are to be scheduled as soon as possible for the week 24 visit.

### Diagnosis and Main Criteria for Inclusion & Exclusion: Criteria for Inclusion:

1. Men and women, 18-75 years (inclusive) of age at screening;
2. Signed informed consent;
3. Must meet the disease specific criteria for HD listed below:
   a. Clinical diagnosis of HD with documented genetic confirmation (Number of CAG repeats ≥40);
   b. Onset of signs and symptoms of disease within the past 2 years and onset between the ages of 18 and 50 years (inclusive); and
   c. UHDRS-TFC score of 7 to 13 (inclusive) at the screening visit. 4. Body Mass Index (BMI) between 20 kg/m² and 32 kg/m² (inclusive);
5. Stable doses of all concomitant medications for 30 days prior to study entry and agree to remain on stable doses for the duration of the study;
6. Negative serum beta-human chorionic gonadotropin (6-hCG) pregnancy result at the screening visit for female participants of childbearing potential; and
7. Willingness to comply with sexual abstinence or contraception guidelines of this study.

### Criteria for Exclusion:

1. Current participation in another clinical trial or completed participation in an interventional trial less than 30 days prior to the screening visit (90 days for a biological treatment);
2. History of gene therapy, cell transplants, or brain surgery aimed at treating the medical condition of interest within 1 year of the screening visit;
3. Mini-mental status examination (MMSE) score≤23 at screening;
4. Prior treatment with trehalose injection, 90.5 mg/mL for intravenous infusion, any other IV trehalose formulation, or known hypersensitivity to trehalose;
5. Current diagnosis and/or healthcare professional-recommended treatment (medication and/or diet) of diabetes mellitus type 1 or type 2;
6. HbA1c≥6.5% at the screening visit;
7. Pregnant or breastfeeding;
8. History of alcohol or drug abuse within the last 2 years;
9. Chronic liver disease including Hepatitis B; Hepatitis C unless successful curative treatment is documented; human immunodeficiency virus (HIV) infection;
10. Prior history of drug-induced liver injury (DILI) and/or laboratory results at screening that indicate inadequate liver function (e.g., ALT, AST, GGT>2 times the upper limit of normal [×ULN]) and/or total bilirubin level>2×ULN);
11. Laboratory results at screening that indicate inadequate renal function (e.g., estimated creatinine clearance of <60 mL/min calculated by the Cockcroft and Gault formula);
12. Any current cardiovascular disease or abnormality on 12-lead electrocardiogram (ECG) at screening that, in the investigator's opinion, is clinically significant and could be a potential safety risk to the participant;
13. Any current psychiatric, neurological, or cognitive disorder that, in the investigator's opinion, may interfere with the participant's ability to provide informed consent or appropriately complete the study's safety or efficacy assessments;
14. Significant suicide risk as indicated by a “yes” response to #4 or #5 under Suicidal Ideation or any “yes” response under Suicidal Behavior on the Columbia Suicide Severity Rating Scale (C-SSRS) during the screening visit; and
15. Any other medical condition or abnormal finding during screening that, in the investigator's opinion, could confound collection or interpretation of safety or efficacy data or be a potential safety risk to the participant.

### Investigational Product, Dosage and Mode of Administration:

Trehalose injection, 90.5 mg/mL for intravenous infusion weight-based dose of 0.75 g/kg will be administered by IV infusion once weekly over a treatment period of 24 weeks.

### Restricted Medications:

There are no known drug-drug interactions with trehalose injection, 90.5 mg/mL for intravenous infusion. Further, oral trehalose or similar molecule (i.e., mannitol) is not permitted during the study.

Participants should not change concomitant medications during the study unless prescribed by a healthcare professional for an exacerbation of a coexisting medical condition or an acute illness. Participants may receive concomitant medications that are medically necessary as standard care to treat symptoms such as adverse events (AEs), inter-current illnesses, and to prevent illness, e.g., vaccines. Over the counter (OTC) medications such as vitamins or herbal supplements are permitted if the participant is taking them at the time of informed consent. The dose and frequency should remain the same throughout the study whenever possible. Participants should not initiate treatment with any other medications or therapeutic devices during their time of participation in the trial. The investigator should be informed if a new treatment is determined to be necessary by a healthcare professional prior to starting the treatment whenever possible.

### Statistical Methods:

Any participant who received at least one dose of trehalose injection, 90.5 mg/mL for intravenous infusion will be included in the safety analysis. AEs, safety laboratory, ECGs, physical exam, concomitant medication, and vital signs data will be presented in tabular format and summarized descriptively, and when appropriate, by visit. No formal hypothesis testing will be carried out on these safety assessments.

C-SSRS responses will be mapped to the Columbia Classification Algorithm of Suicide Assessment (C-CASA) scale, and the frequency distribution of the C-CASA scores will be tabulated by study visit. No hypothesis associated with the C-SSRS or C-CASA scales will be tested.

TABLE 3
Schedule of Assessments

| | Screening | | | | | | | Safety |
|---|---|---|---|---|---|---|---|---|
| | Period | Open-Label Treatment Period | | | | | | Follow-up^{a,b} |
| Assessment/ | Days −14 | | Week | Week | Week | Week | Week | Week |
| Procedure | to −1 | Day 1 | 4 ± 3 d | 8 ± 3 d | 12 ± 3 d | 18 ± 3 d | 24 ± 3 d | 26 ± 3 d |
| Informed Consent | X | | | | | | | |
| Medical History | X | | | | | | | |
| Serum Hepatitis B, | X | | | | | | | |
| C, and HIV testing | | | | | | | | |
| MMSE | X | | | | X^{c} | | X^{c} | |
| PGIC | | | X | X | X | X | X | |
| PGI-S | | X | X | X | X | X | X | |
| CGIC | | | X | X | X | X | X | |
| CGI-S | | X | X | X | X | X | X | |
| HD - UHDRS (TFC | X | | X | X | X | X | X | |
| and independent) | | | | | | | | |
| Hematology | X | | X | | X | | X | |
| Coagulation | X | | | | | | | |
| Serum Chemistry | X | | X | | X | | X | |
| Urinalysis | X | | X | | X | | X | |
| Pregnancy Test | X^{d} | | X | | X | | X | |
| Physical Exam | X | | | | X | | X | |
| Neurological Exam | X | | | | X | | X | |
| 12-Lead ECG | X^{e} | | X | | X | | X | |
| C-SSRS | X | | X | | X | | X | |
| Weight, Height | X^{f} | X | X | | X | X | X | |
| Blood for biomarker | X | | | | X | | X | |
| (neurofilament light | | | | | | | | |
| chain) | | | | | | | | |
| PK Blood Collection | | X^{g} | | | X^{h} | | | |
| Vital Signsⁱ | X | X | Weekly Throughout the Study | | | | | |
| Study Drug | | X | Weekly trehalose injection, 90.5 mg/mL for | | | | | |
| Administration | | | intravenous Infusion | | | | | |
| Adverse Events | | Throughout Study | | | | | | X |
| Concomitant | X | Throughout Study | | | | | | X |
| Medications | | | | | | | | |

Abbreviations: CGIC = Clinical Global Impression of Change, CGI-S = Clinical Global Impression of Severity, C-SSRS = Columbia Suicide Severity Rating Scale, d = days, ECG = electrocardiogram, HIV = human immunodeficiency virus, PGIC = Patient's Global Impression of Change, PGI-S = Patient's Global Impression of Severity PK = pharmacokinetic, SOI = start of infusion, SVC = slow vital capacity, Term = termination.
^{a}Visit can be done via telephone or remote contact only if participant is not continuing on extended treatment;
^{b}To be performed within 2-4 weeks after participant's last dose for participants who withdraw from the study prior to week-24. Participants who discontinue treatment prior to week 24 clinic visit will be encouraged to come back into the clinic for final week 24 assessment;
^{c}If an enrolled participants' MMSE score is ≤23, a study partner/caregiver must be identified to provide consent on behalf of the participant and assist with completion of remaining assessments. Additional assessments may be performed if in the investigator's clinical judgement, the participant's cognition appears to have changed or a change in the participant's cognition or behavior is reported by a family member or care partner;
^{d}Serum pregnancy test at screening only to be performed for female participants of childbearing potential, remaining tests may be urine tests. If at any point during the study a participant is possibly pregnant (or urine test is inconclusive) a urine pregnancy test should be followed by a confirmatory serum pregnancy test.
^{e}Triplicate ECG to be performed at screening only. All other ECGs are single recordings.
^{f}Height is measured only at screening;
^{g}Collect blood samples from all participants for PK analysis at the following time points: pre-dose and 1 hour, 2 hours and 4 hours and 6 hours post SOI;
^{h}Collect blood samples from all participants for PK analysis at the following time points: pre-dose and 1 hour, 2 hours and 4 hours post SOI;
ⁱVital signs are to be measured supine or seated pre-infusion, 30 minutes (±5 mins) after the SOI, 60 minutes after SOI and 90 minutes (±10 mins) after the SOI. If a participant requires a 90-minute infusion duration due to receiving >600 mL of study drug an additional vital signs measurement should be done approximately 30 minutes after the end of infusion.

### List of Abbreviations and Definitions of Terms

The following abbreviations and specialist terms are used in this study protocol. (Table 4)

TABLE 4
Abbreviations and specialist terms.

| Abbreviation | Definition |
|---|---|
| AE | adverse event |
| ALT | alanine aminotransferase |
| AST | aspartate aminotransferase |
| AUC | area under the concentration-time curve |
| ß-hCG | beta-human chorionic gonadotropin |
| BMI | body-mass index |
| BUN | blood urea nitrogen |
| C-CASA | Columbia Classification Algorithm of Suicide Assessment |
| CGIC | Clinical Global Impression of Change |
| CGI-S | Clinical Global Impression of Severity |
| CNS | central nervous system |
| CRF | Case Report Form |
| CRO | Contract Research Organization |
| C-SSRS | Columbia Suicide Severity Rating Scale |
| CYP450 | cytochrome p450 |
| DILI | drug-induced liver injury |
| DSM | Diagnostic and Statistical Manual |
| ECG | electrocardiogram |
| eCRF | electronic Case Report Form |
| EDC | electronic data capture |
| EQVAS | EQ-5D Visual Analog Scale |
| FAS | full analysis set |
| FDA | Food and Drug Administration |
| GCP | Good Clinical Practice |
| GGT | gamma-glutamyl transferase |
| GI | gastrointestinal |
| GLP | Good Laboratory Practice |
| HCT | hematocrit |
| HD | Huntington's disease |
| HGB | hemoglobin |
| HgbA1c | hemoglobin A1c test |
| HIV | human immunodeficiency virus |
| HTT | Huntingtin gene |
| IB | Investigator's Brochure |
| ICF | Informed Consent Form |
| ICH | International Conference on Harmonisation |
| IEC | Independent Ethics Committee |
| INR | international normalized ratio |
| IR-I | Improvement Rate - Investigator |
| IR-P | Improvement Rate - Participant |
| IRB | Institutional Review Board |
| IUD | intrauterine device |
| IV | intravenous |
| LC-3 II | microtubule-associated protein 1A/1B-light chain 3 |
| LDH | lactate dehydrogenase |
| MCH | mean corpuscular hemoglobin |
| MCHC | mean corpuscular hemoglobin concentration |
| MCV | mean corpuscular volume |
| MedDRA | Medical Dictionary for Regulatory Activities |
| mHTT | Mutant huntingtin gene |
| MJD | Machado Joseph Disease |
| MMSE | Mini-Mental Status Exam |
| MTD | maximum tolerated dose |
| m-SARA | Modified Scale for Assessment and Rating of Ataxia |
| OPMD | oculopharyngeal muscular dystrophy |
| OTC | over-the counter |
| PDF | portable document format |
| PGIC | Patient Global Impression of Change |
| PGI-S | Patient Global Impression of Severity |
| PK | pharmacokinetic(s) |
| PKS | pharmacokinetic analysis set |
| PolyA | polyalanine |
| PolyQ | polyglutamine |
| PR | The time from the onset of the P wave to the start |
| | of the QRS complex |
| PT | prothrombin time |
| PTT | partial thromboplastin time |
| QRS | The combination of 3 of the graphical deflections |
| complex | in an electrocardiogram |
| QTc | corrected QT interval |
| QTcF | The QT interval corrected for heart rate using |
| | Fridericia's formula |
| QT | The measure of the time between the start of the |
| interval | Q wave and the end of the T wave |
| RBC | red blood cell |
| rEEC | revised El Escorial criteria |
| ROS | reactive oxygen species |
| RR | The duration of the entire cardiac cycle |
| | (R wave to R wave), measured in seconds |
| SAF | safety set |
| SAE | serious adverse event |
| SAP | Statistical Analysis Plan |
| SARA | Scale for Assessment and Rating of Ataxia |
| SD | standard deviation |
| SI | International System of Units |
| SOC | System Organ Class |
| SOI | start of infusion |
| SUSAR | suspected unexpected serious adverse reaction |
| SVC | slow vital capacity |
| TEAE | treatment-emergent adverse event |
| TFC | total functional capacity |
| TRR-I | Treatment Response Rate - Investigator |
| TRR-P | Treatment Response Rate - Participant |
| UHDRS | Unified Huntington Disease Rating Scale |
| UHDRS- | Unified Huntington Disease Rating Scale - Total |
| TFC | Functional Capacity |
| US | United States |
| WBC | white blood cell |

Investigational Product/Study Drug: Trehalose Injection, 90.5 mg/mL for IV Infusion

When trehalose is administered orally in humans, trehalase enzymes in the brush border of the gut actively cleave trehalose into 2 glucose molecules resulting in <0.5% of trehalose reaching systemic circulation. The most effective way to ensure adequate trehalose exposure in primary therapeutic target organs (brain and muscle) is to circumvent extensive first pass metabolism by administering trehalose as an intravenous (IV) infusion. Trehalose injection, 90.5 mg/mL for intravenous infusion is an injectable formulation of 90.5 mg/mL trehalose for IV infusion developed to circumvent gastrointestinal (GI) metabolism.

### Rationale for Study Endpoints

The primary safety endpoint is the incidences of treatment-emergent adverse events (TEAEs) and serious adverse events (SAEs), including clinically significant laboratory or diagnostic testing abnormalities. This endpoint was selected to establish a safety profile of trehalose injection, 90.5 mg/mL for intravenous infusion within subjected with HD.

Therapeutic endpoints are based on the participant therapeutic response rate (TRR-P) at week 24 [Percentage of participants reporting a treatment response defined as improvement or no change from baseline on the Patient Global Impression of Change (PGIC) & Global Impression of Severity (CGI-S)]. The combination of participant and clinician reported outcomes will allow for a measure of therapeutic efficacy in HD.

### Potential Risks

The safety profile from previous clinical trials of trehalose injection, 90.5 mg/mL for intravenous infusion in adults shows no drug-related AEs other than mild, transient glycosuria, which was asymptomatic and 1 SAE to date, elevated liver enzymes, was considered possibly related to study drug due to temporal association of the changes and lack of alternative causation, however the event resolved without any intervention. Of note, trehalose has no active metabolites—the drug is metabolized by trehalase enzymes into 2 glucose molecules.

Risks of study participation also includes the risk of an infusion reaction or infusion site reactions. There have been no infusion reactions to date and 1 infusion site reaction thus far (infusion site erythema). Toxicology data has suggested that small indwelling catheters may result in vessel changes after multiple infusions. The inflammatory reaction noted was not associated with any generalized inflammation and there was no organ toxicity, thus it was considered a procedural complication of the indwelling catheter. If needed, the use of an implanted venous access device (i.e., Mediport) or percutaneous indwelling catheter is permitted; however, if small caliber indwelling catheters in peripheral vessels is required, they should be for single use only. Additional risks include those risks related to venipuncture (e.g., hematoma, bleeding, pain, infection), which are low, and the risk of progression of disease for participants assigned to receive.

### Potential Benefits

Applicant is conducting this trial with cautious expectations that evaluation of trehalose injection, 90.5 mg/mL for intravenous infusion treatment may show evidence that the drug is able to slow or stabilize the progression of symptoms in participants with HD.

Given the relatively benign safety profile of trehalose injection, 90.5 mg/mL for intravenous infusion and the lack of a highly effective treatment for these diseases, treatment with trehalose injection, 90.5 mg/mL for intravenous infusion for its potential to arrest or slow progression of disease symptoms in participants is medically justified.

### Primary Safety Objective

Determine the safety and tolerability of trehalose injection, 90.5 mg/mL for intravenous infusion in participants with HD.

### Primary Therapeutic Objective

Determine treatment responses of trehalose injection, 90.5 mg/mL for intravenous infusion in participants with HD using a patient-reported outcome measure.

### Secondary Objective

Determine the effectiveness of trehalose injection, 90.5 mg/mL for intravenous infusion on disease progression and severity in participants with HD using clinician- and patient-reported outcome measures.

### Exploratory Objectives

Assess pharmacokinetics (PK) of trehalose injection, 90.5 mg/mL for intravenous infusion in participants with HD.
Assess effect of trehalose injection, 90.5 mg/mL for intravenous infusion treatment on neurofilament light chain concentration in blood.

### Primary Safety Endpoint

Incidences of treatment-emergent adverse events (TEAEs) and serious adverse events (SAEs), including clinically significant laboratory or diagnostic testing abnormalities.

### Primary Therapeutic Endpoint

Treatment Response Rate—Participant (TRR-P) at week 24 [Percentage of participants reporting a treatment response defined as improvement or no change from baseline on the Patient Global Impression of Change (PGIC)].

### Secondary Therapeutic Endpoints

TRR-P at weeks 4, 8, 12 and 18
Treatment Response Rate—Investigator (TRR-I) at weeks 4, 8, 12, 18 and 24 [Percentage of investigators reporting a treatment response defined as participant improvement or no change from baseline on the Clinical Global Impression of Change (CGIC)]
Improvement Rate—Participant (IR-P) at weeks 4, 8, 12, 18 and 24 [Percentage of participants reporting improvement from baseline on the Patient Global Impression of Change (PGIC)]
Improvement Rate—Investigator (IR-I) at weeks 4, 8, 12, 18 and 24 [Percentage of investigators reporting participant improvement from baseline on the Clinical Global Impression of Change (CGIC)]
Change from baseline in Patient Global Impression of Severity (PGIS) at weeks 4, 8, 12, 18, and 24
Change from baseline in Clinical Global Impression of Severity (CGIS) at weeks 4, 8, 12, 18, and 24
Change from baseline in EuroQol visual analogue scale (EQ VAS) score at weeks 4, 8, 12, 18 and 24
Change from baseline in disease specific scale scores for Huntington Disease at weeks 4, 8, 12, 18 and 24
   HD—Unified Huntington Disease Rating Scale (UHDRS)—Total Functional Capacity (TFC) and Independence Scale
TRR-P at weeks 4, 8, 12, 18 and 24 for each disease cohort HD
TRR-I at weeks 4, 8, 12, 18 and 24 for each disease cohort HD

### Exploratory Analyses

Change from baseline in blood neurofilament light chain concentration at weeks 12 and 24 weeks.
Serum PK parameter estimates for trehalose injection, 90.5 mg/mL for intravenous infusion including, but not limited to the area under the curve (AUC), maximum concentration (Cₘₐₓ), time to maximum concentration (Tₘₐₓ) and terminal half-life (t_{1/2}) on day 1 and week 12.

### Overall Study Design

This is an open-label basket study of trehalose injection, 90.5 mg/mL for intravenous infusion for treatment of adults with HD. The study consists of a 2-week screening period, a 24-week open-label treatment period, and a 2-week safety follow-up period. Eligible participants will receive trehalose injection, 90.5 mg/mL for intravenous infusion to be administered as a once weekly IV infusion. The total volume of the infusion will be based on participant weight 0.75 g/kg. The study plans to enroll 10 participants comprised of up to 15 participants with HD.

The primary study objectives are to determine safety and tolerability and treatment responses associated with trehalose injection, 90.5 mg/mL for intravenous infusion in participants with HD. Safety and tolerability will be determined by incidences of TEAEs and serious adverse events SAEs, including clinically significant laboratory or diagnostic testing abnormalities. Laboratory samples, ECG's and C-SSRS are to be obtained at the screening/baseline and week 4, 12 & 24 visits. Physical and neurological examinations are to be conducted at the screening/baseline and week 12 & 24 visits. Vital signs and AEs are to be obtained at visits for study drug infusions and any unscheduled visits. In addition, AEs may be obtained by scheduled or unscheduled telephone communications. Treatment responses will be determined by assessments and comparative analyses of the following general and disease-specific clinical measures that are scheduled to be obtained at the screening/baseline and several specified study visits. All participants are scheduled to complete the PGIC, and all investigators are to complete the CGIC & CGI-S for the respective participant at the week 4, 8, 12, 18 & 24 visits. In addition, all participants are scheduled to complete the PGI-S and EQ-VAS and all investigators are scheduled to complete the CGI-S at the day 1 and week 4, 8, 12, 18 & 24 visits. At the screening/baseline and week 4, 8, 12, 18, & 24 visits, the UHDRS—Independence Scale for participants with HD will be completed.

Blood samples for measurement of neurofilament light chain concentration are scheduled to be obtained from all participants at the screening/baseline and week 12 & 24 visits for analyses of change after initiation of treatment. In addition, blood samples are scheduled to be obtained from all participants at several timepoints during the day 1 and week 12 visits to assess PK of trehalose injection, 90.5 mg/mL for intravenous infusion.

An additional 52 weeks of open-label trehalose injection, 90.5 mg/mL for intravenous infusion treatment is planned for consenting participants who are determined to be medically appropriate after their completion of the primary 24-week open-label treatment period. Participants who do not complete the primary 24-week treatment period or who completed the 24-week treatment period but do not consent to additional treatment are to be scheduled for a safety follow-up visit within 2-4 weeks of their last trehalose injection, 90.5 mg/mL for intravenous infusion dose. All participants who discontinue treatment early and do not plan to continue attending scheduled study visits are to be scheduled as soon as possible for the week 24 visit.

### Duration of Treatment

The total duration of the study is up to 28 weeks for an individual participant. This includes a 2-week screening period, a 24-week open-label treatment period and 2-week safety follow-up. Participants who complete the 24-week treatment period may be eligible to participate in an additional 52 weeks of open-label treatment of trehalose injection, 90.5 mg/mL for intravenous infusion.

### Study Population

### Number of Participants

The study plans to enroll up to 15 participants with HD.

### Participant Inclusion Criteria and Participant Exclusion Criteria

Same as described above.

### Restricted Medications

There are no known drug-drug interactions with trehalose injection, 90.5 mg/mL for intravenous infusion; however, the following restrictions should be followed during the course of the study:
There are no known drug-drug interactions with trehalose.
Oral trehalose or similar molecules (i.e., mannitol) are not permitted during the study.
Participants should not change concomitant medications during the study unless they experience an exacerbation or an acute illness. Participants may receive concomitant medications that are medically necessary as standard care to treat symptoms such as AEs, inter-current illnesses, and to prevent illness, e.g., vaccines.
Over the counter (OTC) medications such as vitamins or herbal supplements are permitted if the participant is taking them at the time of consent. The dose and frequency should remain the same throughout the study whenever possible.
Participants should not initiate treatment with any other medications or therapeutic devices during their time of participation in the trial. The investigator should be informed if a new treatment is determined to be necessary by a healthcare professional prior to starting the treatment whenever possible.

### Trehalose Administration

The participant will receive weekly IV infusions of study drug trehalose injection, 90.5 mg/mL for intravenous infusion for 60±5 minutes for volumes≤600 mL or 90±5 minutes for volumes>600 mL) throughout the study. All infusions will be administered using an infusion pump and will be followed with a saline IV flush.

### Concomitant Medications

To date, there are no known pharmacokinetic drug interactions with trehalose injection, 90.5 mg/mL for intravenous infusion. Trehalose injection, 90.5 mg/mL for intravenous infusion has no effect on the cytochrome p450 (CYP450) system; therefore, participants may continue their present medications as long as their treatment regimen is unchanged, and the medications are not restricted medications (Section 0).

Investigators may prescribe concomitant medications or treatments to provide adequate supportive care as deemed necessary provided they are not restricted medications (Section 0). The medical monitor should be contacted if there are any questions regarding concomitant or prior therapy.

Participants should not change concomitant medications during the study unless they experience a new medical condition, exacerbation of an existing medical condition requiring treatment or an acute illness. Participants may receive concomitant medications that are medically necessary as standard care to treat symptoms such as, AEs, inter-current illnesses, anxiety, and to prevent illness, e.g., vaccines.

OTC medications such as vitamins or herbal supplements are permitted if the participant is taking them at the time of consent. The dose and frequency should remain the same throughout the study if at all possible.

### Study Assessments

The procedures and assessments that will be conducted during this study are described in this section and summarized in the Schedules of Assessments (Table). Detailed instructions regarding all laboratory procedures, including collection and handling of samples, will be included in the study laboratory manual provided by the sponsor or its designee.

To ensure consistency in administration and interpretation of subjective clinical outcome measures, site staff/raters will be provided training or certification as appropriate. Written informed consent must be granted by each participant or their legally authorized representative prior to the initiation of any study procedure or assessment (other than those considered standard of care).

### Sequence of Assessments

Assessments should be conducted in a sequence which allows for best clinical practice and participant experience while also ensuring protocol compliance.

During study visits it is recommended that the Mini-Mental Status Exam, efficacy assessments [PGIC, PGI-S, CGIC, CGI-S, EQ-VAS and disease specific assessments (i.e., ALSFRS-R, SVC, bulbar function scale, UHDRS, m-SARA)] should be completed first followed by safety assessments (C-SSRS, physical exam, neurological exam, 12-lead ECG, and laboratory blood/urine collection). Both efficacy and safety assessments should be completed prior to trehalose injection, 90.5 mg/mL for intravenous infusion to minimize confounding factors.

### Screening Assessments

Participants who do not pass a screening assessment (e.g., not achieving acceptable limits in the clinical safety laboratory tests at screening) may be re-screened once at least 30 days after the original screen failure at the investigator's discretion. The screening period may be up to 2 weeks in duration, but participants can be enrolled upon completion of all screening assessments.

### Demographic/Medical History

A review of demographic parameters, including age, gender, race, and ethnicity will be performed at screening. Family history of related disease will also be recorded if known.

Past and present medical and surgical history will be recorded according to the Schedule of Assessments (Table). Any ongoing condition and signs and symptoms observed prior to the initiation of study treatment should be recorded as past medical history.

Disease specific history will be recorded and includes date of diagnosis, primary symptoms at time of diagnosis, disease progression history, and genetic confirmation documentation.

### Weight and Height

Weight will be measured as per the Schedules of Assessments (Table), to re-assess and adjust the dose if necessary. No bulky clothes or shoes should be worn. Doses should only be adjusted based on changes in weight (2 kg increase or decrease) noted during clinical trial assessments.

Height will be measured only at screening for calculation of body-mass index (BMI).

### Screening Vital Signs

Vital signs at screening will include temperature, blood pressure, respiration rate, and heart rate. Resting vital signs will be measured supine (seated, if supine is not possible) with legs uncrossed, back and arms supported following at least 5 min rest.

### Screening ECG

Triplicate 12-lead ECGs will be performed at screening and reviewed by designated a cardiologist. ECGs should be performed prior to blood collection.

Three (3) serial, resting, and supine 12-lead ECGs will be conducted within 10 minutes total time. The mean of the triplicate ECG measurements performed at screening, including Fridericia corrected QT (QTcF), will serve as the participant's baseline QTcF value for all post-dose comparisons.

ECG parameters to be evaluated include heart rate, the time from the onset of the P wave to the start of the QRS complex (PR), the duration of the entire cardiac cycle, measured in seconds (RR), the combination of 3 of the graphical deflections (QRS complex), the measure of the time between the start of the Q wave and the end of the T wave (QT) intervals, and the QT interval corrected for heart rate using Fridericia's formula (QTcF).

### Cognitive Performance Assessment

The Mini-Mental Status Exam (MMSE) will be administered by a qualified clinician to assess cognitive performance within the orientation, attention, memory, language, and visual spatial skills domain. The MMSE will be administered at the time of screening, and on weeks 12 and 24 to assess the participants' capacity to consent. If an enrolled participants' MMSE score is less than or equal to 23 on weeks 12 or 24, a person responsible consent form will be used to obtain consent for remaining study activities and a study partner/caregiver will be identified to assist with completion of remaining assessments.

### Efficacy Assessments

### Clinician Global Impression of Change (CGIC) & Patient Global Impression of Change (PGIC)

The CGIC & PGIC are two distinct questionnaires, one completed by the evaluating Investigator (CGIC), and one completed by the participant (PGIC).

The CGIC records the physician investigator's overall assessment of change in the participant's current disease status compared to their baseline assessment.

The PGIC is a patient-reported assessment. The participant records how they feel their medical condition is doing overall now compared to how they felt it was doing overall before starting study treatment.

If applicable care giver impression of change will also be reported as part of the PGIC.

### Clinical Global Impression of Severity (CGI-S) and Patient Global Impression of Severity

The CGI-S & PGI-S are two distinct questionnaires, one completed by the evaluating Investigator (CGI-S), and one completed by the study participant (PGI-S).

The CGI-S is 5-point scale that requires the clinician to rate the overall severity of the participant's disease at the time of assessment relative to the clinician's past experience with participants who have the same diagnosis.

The PGI-S is 5-point scale that requires the participant to rate the overall severity of their disease at the time of assessment relative to their past experiences.

### Slow Vital Capacity (SVC)

SVC using spirometry will be used to assess the treatment effect on respiratory muscle function. SVC measures the volume that can be exhaled from a full inhalation after exhaling to a maximum as slowly as possible. SVC analysis will include SVC, predicted SVC and % of predicted SVC. This test should be conducted by a qualified technician and ideally repeated when possible, the same technician and equipment throughout the study. Calibration of the spirometry equipment must be completed in accordance with user guides and spirometry manual.

The assessment of SVC at the Screening visit is used to determine initial participant suitability for the study.

### Center for Neurologic Study Bulbar Function Scale (CNS-BFS)

The CNS-BSF is a self-report questionnaire consisting of three domains (swallowing, speech, and salivation).

### Unified Huntington's Disease Rating Scale—(UHDRS-TFC)

The UHDRS is a research tool developed to provide a uniform assessment of the clinical features and course of HD. For this study we are focusing on the functional capacity domain which includes the Huntington's Disease functional capacity scale, scored on a 0- to 2- or 3-point scale, and the Independence Scale, scored from 10 to 100.

### EQ-5D Visual Analog Scale (EQ-VAS)

The EQ-VAS records the participant's self-rated health status on a vertical visual analogue scale, where the endpoints are labelled ‘The best health you can imagine’ and ‘The worst health you can imagine’. The EQ-VAS is used as a quantitative measure of health status and treatment outcome that reflect the participant's own judgement.

### Exploratory Assessments

### Pharmacokinetic Assessments

A population PK approach or non-compartmental analysis, whichever is most appropriate, will be used in this study. Participants will have blood collected for PK analysis at:
Day 1, all participants will have blood collected for PK analysis at the following time points: pre-dose and 1 hour, 2 hours, 4 hours, and 6 hours post start of infusion (SOI).
At Week 12, all participants will have blood drawn for PK analysis at the following time points: pre-dose, and 1 hour, 2 hours and 4 hours post SOI.
   Biomarkers Associated with Autophagy

Blood/serum samples will be obtained from participants to detect neurofilament light chain concentration. Details regarding sample collection, preparation, and shipment will be provided in the laboratory manual.

### Safety Assessments

### Vital Signs

Vital signs will include temperature, blood pressure, respiration rate, and heart rate. Resting vital signs will be measured supine (seated, if supine is not possible) with legs uncrossed, back and arms supported following at least 5 min rest. Vitals signs will be measured for all infusions at the following 4 time points (Error! Reference source not found.):
1. Pre-infusion
2. 30 minutes (±5 minutes) post SOI
3. 60 minutes (±5 minutes) post SOI
4. 90 minutes (±10 minutes) post SOI
5. If a participant requires a 90-minute infusion duration due to receiving >600 mL of study drug an additional vital signs measurement should be done 30 minutes (±10 minutes) after the end of infusion.

### Columbia Suicide Severity Rating Scale

The Columbia Suicide Severity Rating Scale (C-SSRS) is a unique, simple, and short method of assessing both behavior and ideation that tracks all suicidal events and provides a summary of suicidal ideation and behavior. It assesses the lethality of attempts and other features of ideation (frequency, duration, controllability, reasons for ideation, and deterrents), all of which are significantly predictive of completed suicide.

Two versions of the scale will be used: a ‘screening’ version (lifetime assessment and recent history, used at screening) and a ‘since last visit’ version (used on all subsequent occasions).

A qualified rater will complete the scale based on the participants' responses, which will be used as the source document.

### Adverse and Serious Adverse Events

### Definition of Adverse Events (AEs)

An adverse event (AE) is defined as any untoward medical occurrence associated with a study drug in humans or study procedure regardless of whether the event is assessed as related to the study drug or procedure.

An AE (also referred to as an adverse experience) can therefore be any unfavorable and unintended sign (e.g., an abnormal laboratory finding), symptom, or disease temporally associated with the use of a drug, without any judgment about causality or seriousness. An AE can arise from any use of the drug (e.g., off-label use, use in combination with another drug) and from any route of administration, formulation, or dose, including an overdose. An abnormal result of diagnostic procedures including abnormal laboratory findings will be considered an AE if it fulfills one or more of the following:
Results in participant's withdrawal by the investigator;
Is associated with an SAE;
Is associated with clinical signs or symptoms;
Requires a medical or surgical intervention;
Is considered by the physician to be of clinical significance (a laboratory abnormality that is not clinically significant will not be considered an AE).

A new condition or the worsening of a pre-existing condition (either an increase in frequency or intensity) will be considered an AE.

An adverse reaction means any AE caused by a drug. Adverse reactions are a subset of all suspected adverse reactions for which there is reason to conclude that the drug caused the event.

A suspected adverse reaction is any AE for which there is a reasonable possibility that the drug caused the AE.

### AEs do not Include the Following:

Stable or intermittent chronic conditions (such as myopia requiring eyeglasses) that are present prior to study entry and do not worsen during the study
Medical or surgical procedures (e.g., surgery, endoscopy, tooth extraction, transfusion); the condition that leads to the procedure is an AE if not present at baseline
Overdose of either study drug or concomitant medication without any signs or symptoms unless the participant is hospitalized for observation
Hospitalization for elective surgery planned prior to the study (situation where an untoward medical occurrence has not occurred)

AEs will be collected continuously starting from the signing of the informed consent form (ICF) through the last study visit. Investigators are instructed to follow any AEs through to their conclusion. In the event of participant withdrawal from the study, AE monitoring should continue through the last study visit, if possible.

Participants should be counseled that any new side effect occurring between scheduled visits should be brought to the attention of the investigator.

### Assessment of AEs by the Investigator

All AEs, whether observed by the investigator or designee or volunteered by or elicited from the participant, should be recorded. AEs will be recorded from the time a participant has signed the ICF and throughout the study, including the follow up period. Severity of the AE will be assessed by the investigating physician in accordance with the definitions below.

### Assessment of Severity

Three definitions only should be used by the investigating physician to describe the intensity (severity) of the AE (Table 5). Only one severity definition should be used for each AE (e.g., “mild/moderate” is not acceptable).

TABLE 5
Definition of Adverse Events Severity

| SEVERITY | DEFINITION |
|---|---|
| MILD | A mild AE is one where the symptoms are barely noticeable to the |
| | participant. It does not influence the performance or prevent the participant |
| | from carrying on with normal life activities. |
| MODERATE | A moderate AE is one where the symptoms make a participant |
| | uncomfortable and cause some impairment to normal life activities. |
| | Treatment for symptom(s) may be required. |
| SEVERE | A severe AE is one where the symptoms cause severe discomfort to the |
| | participant and severely limit the participant's normal daily activities. |
| | Treatment for symptom(s) is given. |

### Assessment of Causality

The investigator will document in his/her opinion the relationship of the AE to the study drug using the criteria outlined in Table 6.

TABLE 6
Definition of Adverse Events Relationship to Study Drug

| CAUSALITY | DEFINITION | CLARIFICATION |
|---|---|---|
| Unrelated | In general, this | An adverse experience may be considered unrelated to the study |
| | category can be | drug if or when (must have 2): |
| | considered applicable | It does not follow a reasonable temporal sequence from the |
| | to those AE, which | administration of the test drug. |
| | after careful medical | It could readily have been produced by the participant's clinical |
| | consideration at the | state, environmental or toxic factors, or other modes of therapy |
| | time they are | administered to the participant. |
| | evaluated, are judged | It does not follow a known pattern of response to the test drug. |
| | to be unrelated to the | It does not reappear or worsen when the drug is re-administered. |
| | test drug. | |
| Possibly Related | This category applies | An adverse experience may be considered possibly related to the |
| | to those adverse | study drug if or when (at least 2 of the following): |
| | events for which, after | It follows a reasonable temporal sequence from administration |
| | careful medical | of the drug. |
| | consideration at the | It could not readily have been produced by the participant's |
| | time they are | clinical state, environmental or toxic factors, or other modes of |
| | evaluated, a | therapy administered to the participant. |
| | connection with the | It follows a known pattern of response to the test drug. |
| | test drug | |
| | administration | |
| | appears unlikely but | |
| | cannot be ruled out | |
| | with certainty. | |
| Probably | This category applies | An adverse experience may be considered probably related to |
| Related | to those adverse | the study drug if or when (at least 3 of the following): |
| | events which, after | It follows a reasonable temporal sequence from administration |
| | careful medical | of the drug. |
| | consideration at the | It could not be reasonably explained by the known |
| | time they are | characteristics of the participant's clinical state, environmental |
| | evaluated, are felt | or toxic factors or other modes of therapy administered to the |
| | with a high degree of | participant. |
| | certainty to be related | It disappears or decreases on cessation or reduction in dose. |
| | to the test drug. | There are important exceptions when an adverse event does not |
| | | disappear upon discontinuation of the drug, yet drug-relatedness |
| | | clearly exists. |
| | | It follows a known pattern of response to the test drug. |

### Assessment of Outcome

Outcome to date is classified as follows:
Recovered—The participant has fully recovered from the AE with no residual effect observable
Recovered with sequelae—The participant has recovered from the AE with residual effects observable
Recovering—The participant status is improved but has not yet been recovered
Ongoing—AE is not recovered
Fatal—The subject has died as a consequence of the event. Date of death is recorded as stop date for the AE
Unknown

### Disease Progression

Disease progression can be considered as a worsening of a participant's condition that is being studied. It may be reflected by an increase in the severity of the condition or an increase in the symptoms. Disease progression that is measured as an outcome measure should not be reported as an AE.

### Data Analysis/Statistical Methods

This section summarizes the planned approaches and statistical methodology to be used for the analysis and presentation of the study data. A detailed description of the planned analysis and statistical methods for the study will be documented in the Statistical Analysis Plan (SAP) which will be finalized prior to database lock. The SAP will supersede the plans outlined in the protocol; however, any major modifications of the primary endpoint definition and/or its analysis will be reflected in a protocol amendment. Furthermore, any deviations from the planned analyses described in the SAP will be described and justified in the final clinical study report.

Continuous variables will be summarized descriptively by visit showing number of evaluable participants, mean, standard deviation, median, quartiles, minimum, and maximum. Additionally, the same descriptive statistics will be provided for changes from baseline at each post-baseline visit, when appropriate. Categorical variables will be summarized using number evaluable, frequencies and percentages.

The study includes a 24-week open-label treatment period. After Week 24, participants will be invited to participate in an open-label extension. The therapeutic and safety analyses of all endpoints will be conducted on the data collected through Week 24 (end of the treatment period).

### Sample Size Considerations

The study plans to enroll up to 15 participants with HD.

### Analysis Populations

The following analysis populations will be used for all analyses and presentations:
The full analysis set (FAS) includes all enrolled participants who receive at least 1 dose of study medication and have at least one post-baseline efficacy assessment.
The safety set (SAF) includes all participants who receive any study medication.
The PK analysis set (PKS) includes all participants in the SAF for whom a sufficient number of plasma trehalose concentrations are available to allow for PK analysis.

The SAF will be used for the primary safety analyses while the FAS will be used for the therapeutic and secondary endpoints. The PKS will be used for the Population PK or non-compartmental analysis.

### Statistical Methods

### Demographics, Baseline Characteristics, and Participant Disposition

Demographics and other baseline characteristics will be summarized using descriptive statistics for the SAM and FAS populations.

Subject disposition will be summarized and will include numbers screened, enrolled, dosed, withdrawn prior to completing the treatment period, completing the treatment period.

The number and percent of participants in each analysis population will be presented. The primary reason for study discontinuation will be summarized.

### Safety Analyses

Safety evaluations will include analysis of:

### Safety Endpoint

Incidences of treatment-emergent adverse events (TEAEs) and serious adverse events (SAEs), including clinically significant laboratory or diagnostic testing abnormalities

The SAF population will be used for all safety analyses. AEs, SAEs, safety laboratory, ECGs, physical exam, concomitant medication, and vital signs data will be presented in tabular format and summarized descriptively, and when appropriate, by visit. No formal hypothesis testing will be carried out on these safety assessments.

C-SSRS responses will be mapped to the Columbia Classification Algorithm of Suicide Assessment (C-CASA) scale, and the frequency distribution of the C-CASA scores will be tabulated by study visit. No hypothesis associated with the C-SSRS or C-CASA scales will be tested.

A number of embodiments of the invention have been described. Nevertheless, it will be understood that various modifications may be made without departing from the spirit and scope of the disclosure. Accordingly, other embodiments are within the scope of the following claims.

### Example 3. Treatment of Adults with HD Using IV Trehalose

This study consisted of a 2-week screening period, a 24-week open-label treatment period, and a 2-week safety follow-up period. Eligible subjects received substantially purified trehalose, administered as a once weekly IV infusion over 60 minutes±5 min for volumes≤600 mL or 90 minutes±5 min for volumes>600 mL. The total volume of the infusion was based on the weight of the subject and a dose of 0.75 g trehalose per kg of subject weight (g/kg). An additional 52 weeks of open-label trehalose treatment was planned for consenting subjects who were determined to be medically appropriate after their completion of the primary 24-week open-label treatment period.

The primary study objectives were to determine safety and tolerability, and treatment responses associated with trehalose administered to subjects with HD. Safety and tolerability were determined by incidences of treatment-emergent adverse events (TEAEs) and serious adverse events (TESAEs), including clinically significant laboratory or diagnostic testing abnormalities. Laboratory samples, electrocardiograms (ECGs), and a rating on the Columbia Suicide Severity Rating Scale (C-SSRS) were obtained from the subject at the screening/baseline and during week 4, week 12, and week 24. Physical and neurological examinations of the subject were conducted at the screening/baseline and during week 12 and week 24. Vital signs and adverse events (AEs) of the subject were obtained at visits for study drug infusions and any unscheduled visits. In addition, AEs of the subject were obtained by scheduled or unscheduled telephone communications. Treatment responses were determined by assessments and comparative analyses of the following clinical scores that were obtained at the screening/baseline and follow-up study visits. All subjects completed the Patient Global Impression of Change (PGIC), the Clinical Global Impression of Change (CGIC), and the Clinical Global Impression of Severity (CGI-S) during week 4, week 8, week 12, week 18, and week 24. In addition, all participants completed the Patient's Global Impression of Severity (PGI-S), EQ Visual Analog Scale (EQ-VAS), and the CGI-S on day 1 and during week 4, week 8, week 12, week 18, and week 24. At the screening/baseline and during week 4, week 8, week 12, week 18, and week 24, subjects completed the UHDRS—TFC and the UHDRS—Independence Scale.

Blood samples were obtained from subjects at the screening/baseline and during week 12 and week 24 for analyses. Blood serum was to determine the concentration of neurofilament light chain (NfL) protein in blood serum before and after initiation of trehalose treatment. Nfl concentrations in blood serum were measured using a Simoa® NF-light™ Advantage Kit by Quanterix. Higher concentrations of Nfl in subject blood serum is an indicator of clinical severity of HD, and lower concentrations compared to concentrations of the same subject indicates reversal or slowing of HD progression. The NfL blood serum concentrations of one subject are provided in Table 7.

TABLE 7
NfL blood serum concentration at various time points.

| Subject | Time | NfL Protein Concentration (pg/mL) |
|---|---|---|
| 02-001 (HD) | Screening / Day 0 | 21.20 |
| 02-001 (HD) | Week 12 / Day 84 | 17.55 |
| 02-001 (HD) | Week 24 / Day 168 | 19.95 |

Furthermore, in 3 months (12 weeks) of observation, there was no change in the UHDRS Independence Scale or Functional Capacity in the subject with HD, the mini-mental status examination (MMSE) improved by one point (from 26 to 27), the CGIC and CGI-S were rated as “A Little Better”, and the Global impression of Health on a Visual Analog Scale improved from a 70 at baseline to a 90.

What is claimed is:

1. A method of treating Huntington's disease in a subject in need thereof, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

2. A method of alleviating one or more symptoms of Huntington's disease in a subject in need thereof, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

3. The method of claim 2, wherein the one or more symptoms are selected from the group consisting of: behavioral disturbances, clumsiness, moodiness, irritability, paranoia, apathy anxiety, hallucinations, abnormal eye movements, depression, impaired ability to detect odors, dystonia, involuntary movements, trouble with balance and walking, chorea with twisting and writhing motions, unsteady gait (style of walking), slow reaction time, general weakness, weight loss, dysarthria (speech difficulties), stubbornness, rigidity (continual tension of the muscles), bradykinesia (difficulty initiating and continuing movements), severe chorea, serious weight loss, inability to speak, inability to walk, swallowing problems, inability to care for oneself, and combinations thereof.

4. A method of treating Huntington's disease in a subject in need thereof, comprising intravenously administering an aqueous pharmaceutical formulation to the subject, wherein the formulation comprises substantially purified trehalose as the sole active ingredient, wherein the subject has a UHDRS-TFC score of 7 to 13 prior to the administration.

5. A method of alleviating one or more symptoms of Huntington's disease in a subject in need thereof, comprising intravenously administering an aqueous pharmaceutical formulation to the subject, wherein the formulation comprises substantially purified trehalose and a trehalase inhibitor as the sole active ingredients, wherein the subject has a UHDRS-TFC score of 7 to 13 prior to the administration.

6. A method of treating Huntington's disease in a subject in need thereof, comprising:
identifying a subject as having Huntington's disease; and
intravenously administering an aqueous pharmaceutical formulation to the subject, wherein the formulation comprises a single active ingredient consisting essentially of substantially purified trehalose, and
wherein the subject has a UHDRS-TFC score of 7 to 13 prior to the administration.

7. A method of treating Huntington's disease in a subject in need thereof, comprising:
identifying a subject as having Huntington's disease; and
intravenously administering to the subject an aqueous pharmaceutical formulation comprising substantially purified trehalose and a trehalase inhibitor as the sole active ingredients, and
wherein the subject has a UHDRS-TFC score of 7 to 13 prior to the administration.

8. The method of any one of claim 4, 5, or 7, wherein the trehalase inhibitor is selected from the group consisting of: validimycin A, trehazolin, amygdalin; or a combination of any of the foregoing; or a pharmaceutically acceptable salt of any of the foregoing.

9. The method of any one of claims 1-8, further comprising:
determining a first UHDRS-TFC score for the subject at a first time point;
determining a second UHDRS-TFC score for the subject at a second time point;
wherein the second UHDRS-TFC score is greater than or equal to the first UHDRS-TFC score; and
wherein the first UHDRS-TFC score is determined prior to the first administration of the pharmaceutical formulation and the second UHDRS-TFC score is determined after the first administration of the pharmaceutical formulation.

10. The method of claim 9, wherein the second UHDRS-TFC score is about 1 and about 10 points higher than the first UHDRS-TFC score.

11. The method of claim 10, wherein the second UHDRS-TFC score is about the same as the first UHDRS-TFC score.

12. The method of any one of claims 10-11, wherein the first UHDRS-TFC score is determined no more than seven days prior to the first administration of the pharmaceutical formulation.

13. The method of any one of claims 9-12 wherein the first UHDRS-TFC score is determined no more than three days prior to the first administration of the pharmaceutical formulation.

14. The method of any one of claims 9-13, wherein the second UHDRS-TFC score is determined at about 13 to about 52 weeks after the first UHDRS-TFC score.

15. The method of any one of claims 9-14, wherein the second UHDRS-TFC score is determined at about 13, about 26, about 39, or about 52 weeks after the first UHDRS-TFC score.

16. The method of any one of claims 9-15, wherein the second UHDRS-TFC score is determined about 52 weeks after the first UHDRS-TFC score.

17. The method of any one of claims 9-16, wherein the first UHDRS-TFC score is determined no more than three days prior to the first administration of the pharmaceutical formulation, wherein the second UHDRS-TFC score is determined about 52 weeks after the first UHDRS-TFC score, and wherein the second UHDRS-TFC score is greater than or equal to the first UHDRS-TFC score.

18. The method of claim 17, wherein the second UHDRS-TFC score is from 1 to 10 points higher than the first UHDRS-TFC score.

19. The method of claim 18, wherein the second UHDRS-TFC score is about the same as the first UHDRS-TFC score.

20. The method of any one of claims 9-19, wherein the second UHDRS-TFC score is determined about 13 weeks after the first UHDRS-TFC score, and wherein the second UHDRS-TFC score is greater than or equal to the first UHDRS-TFC score.

21. A method of reducing the rate of decrease in UHDRS-TFC score in a subject having Huntington's disease, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.

22. A method of reducing the rate of decrease in UHDRS-TFC score in a subject having Huntington's disease, comprising administering intravenously to the subject an aqueous pharmaceutical formulation comprising substantially purified trehalose and a trehalase inhibitor as the sole active ingredients; wherein the subject has a UHDRS-TFC score of 7 to 13 prior to the administration.

23. The method of claim 22, wherein the trehalase inhibitor is selected from the group consisting of: validimycin A, trehazolin, amygdalin; or a combination of any of the foregoing; or a pharmaceutically acceptable salt of any of the foregoing.

24. The method of any one of claims 21-23, wherein the rate of decrease in UHDRS-TFC score is reduced by about 10% to about 95%.

25. The method of any one of claims 21-24, wherein the rate of decrease in UHDRS-TFC score is reduced by about 10% to about 50%.

26. The method of any one of claims 21-25, wherein the rate of decrease in UHDRS-TFC score is reduced by about 10% to about 25%.

27. The method of any one of claims 1-26, further comprising:
determining a first EuroQol visual analog scale (EQ-VAS) score for the subject at a first time point;
determining a second EuroQol visual analog scale (EQ-VAS) score for the subject at a second time point;
wherein the second EQ-VAS score is greater than the first EQ-VAS score; and
wherein the first EuroQol-5D-3L score is determined prior to the first administration of the pharmaceutical formulation and the second EuroQol-5D-3L score is determined after the first administration of the pharmaceutical formulation.

28. The method of claim 27, wherein the second EQ-VAS score is determined after the first administration of the pharmaceutical formulation.

29. The method of claim 27 or 28, wherein the second EQ-VAS score is greater than the first EQ-VAS score.

30. The method of any one of claims 27-29, wherein the first EQ-VAS score is determined prior to the first administration of the pharmaceutical formulation.

31. The method of any one of claims 27-30, wherein the first EQ-VAS score is determined no more than seven days prior to the first administration of the pharmaceutical formulation.

32. The method of any one of claims 27-31, wherein the first EQ-VAS score is determined no more than three days prior to the first administration of the pharmaceutical formulation.

33. The method of any one of claims 27-32, wherein the second EQ-VAS score is determined at about 13, about 26, about 39, or about 52 weeks after the first EQ-VAS score.

34. The method of any one of claims 27-33, wherein the second EQ-VAS score is determined at about 24 or 52 weeks after the first EQ-VAS score.

35. The method of any one of claims 27-34, wherein the first EQ-VAS score is determined no more than three days prior to the first administration of the pharmaceutical formulation, wherein the second EQ-VAS score is determined at about 52 weeks after the first EQ-VAS score, and wherein the second EQ-VAS score is greater than the first EQ-VAS score.

36. The method of any one of claims 27-35, wherein the second EQ-VAS score is determined about 24 weeks after the first EQ-VAS score, and wherein the second EQ-VAS score is greater than the first EQ-VAS score.

37. The method of any one of claims 1-36, further comprising administering one or more additional therapeutic agents to the subject.

38. The method of claim 37 wherein the one or more additional therapeutic agents are independently selected from: acetazolamide (Diamox), 4-amantadine, aminopyridine, armodafinil (Nuvigil), atomoxetine (Strattera), baclofen, botulinum toxin shots, buproprion (Wellbutrin), buspirone (Buspar), carbamazepine, carbidopa-levodopa, carnitine, clonazepam, creatine, cymbalta, dantrolene sodium (Dantrium), desvenlafaxine (Pristiq), diazepam (Valium), flunarizine, gabapentin (Neurontin), isoniazid, levetiracetam, levodopa, lyrica, meclizine, memantine, modafinil (Provigil), NAC (N-acetylcysteine), ondansetron (Zofran), pramipexole (Mirapex), primidone, propranolol, pyridostigmine, riluzole (Rilutek), ropinirole (Requip), scopolamine, selective norepinephrine-serotonin reuptake inhibitors, selective serotonin reuptake inhibitors, selegiline (Eldepryl), sinemet, tizanidine (Zanaflex), topiramate, trihexyphenidyl, valproic acid (Depakote), venlafaxine (Effexor), or a pharmaceutically acceptable salt of any of the foregoing.

39. The method of claim 37 or 38, wherein the one or more additional therapeutic agent is administered simultaneously or sequentially in either order with the pharmaceutical formulation.

40. A method of decreasing soluble mutant huntingtin protein (mHTT) levels in a subject having Huntington's Disease, comprising intravenously administering an aqueous pharmaceutical formulation to the subject, wherein the formulation comprises a single active ingredient consisting essentially of substantially purified trehalose, wherein the subject has a UHDRS-TFC score of 7 to 13 prior to the administration.

41. A method of decreasing soluble mutant huntingtin protein (mHTT) levels in a subject having Huntington's Disease, comprising administering intravenously to the subject an aqueous pharmaceutical formulation comprising substantially purified trehalose and a trehalase inhibitor as the sole active ingredients; wherein the subject has a UHDRS-TFC score of 7 to 13 prior to the administration.

42. The method of any one of claims 1-41, wherein the Huntington's disease is or juvenile onset Huntington's disease.

43. The method of any one of claims 1-41, wherein the Huntington's disease is or adult onset Huntington's disease.

44. A method of stabilizing or decreasing the concentration of neurofilament light chain (NfL) polypeptide in blood serum of a subject having previously been diagnosed with HD, comprising intravenously administering an aqueous pharmaceutical formulation to the subject;
wherein the formulation comprises a single active ingredient consisting essentially of substantially purified trehalose; and
wherein the subject has a UHDRS-TFC score of 7 to 13 prior to the administration.
